SEQUENCE LISTING (158 sequences)

SEQ ID NO: 1 - RNA, 80 nt, Artificial
  RNA aptamer
gggacgcucacguacgcucaccggguuauuggcgcaauauugguauccuguauuggucugucagugccuggacgugcagu

SEQ ID NO: 2 - RNA, 80 nt, Artificial
  RNA aptamer
gggacgcucacguacgcucacguccgaucgauacugguauauuggcgccuucguggaaugucagugccuggacgugcagu

SEQ ID NO: 3 - RNA, 80 nt, Artificial
  RNA aptamer
gggacgcucacguacgcucaccuguuuugucuagguuuauuggcgcuuauuccuggaaugucagugccuggacgugcagu

SEQ ID NO: 4 - RNA, 80 nt, Artificial
  RNA aptamer
gggacgcucacguacgcucacucaggugauuggcgcuauuuaucgaucgauaauugaaugucagugccuggacgugcagu

SEQ ID NO: 5 - RNA, 80 nt, Artificial
  RNA aptamer
gggacgcucacguacgcucauguuccuuuggguuauuggcuccuuguugaccaggggaugucagugccuggacgugcagu

SEQ ID NO: 6 - RNA, 80 nt, Artificial
  RNA aptamer
gggacgcucacguacgcucacaacacucgaaggguuuauuggccccaccaugguggaaugucagugccuggacgugcagu

SEQ ID NO: 7 - RNA, 80 nt, Artificial
  RNA aptamer
gggacgcucacguacgcucacgguuauuggcggaggaucugucauggcaugccucgacugucagugccuggacgugcagu

SEQ ID NO: 8 - RNA, 80 nt, Artificial
  RNA aptamer
gggacgcucacguacgcucacuucuuucccacucacgucucgguuuuauugguccaguuuucagugccuggacgugcagu

SEQ ID NO: 9 - RNA, 80 nt, Artificial
  RNA aptamer
gggacgcucacguacgcucaggugaauuggcacuucuuuaucuacggaucgagucggaugucagugccuggacgugcagu

SEQ ID NO: 10 - RNA, 67 nt, Artificial
  RNA aptamer
gggacgcucacguacgcucagguuuauuggugccguguaguggaauaucagugccuggacgugcagu

SEQ ID NO: 11 - RNA, 80 nt, Artificial
  RNA aptamer
gggacgcucacguacgcucacuucccuagacccuccagguuacaggcgccgcccggaaugucagugccuggacgugcagu

SEQ ID NO: 12 - RNA, 56 nt, Artificial
  RNA aptamer
ggguacgcucagguauauuggcgccuucguggaaugucagugccuggacgugcagu

SEQ ID NO: 13 - RNA, 54 nt, Artificial
  RNA aptamer
ggguacgcucagguauauuggcgccucgggaaugucagugccuggacgugcagu

SEQ ID NO: 14 - RNA, 37 nt, Artificial
  RNA aptamer
ggguauauuggcgccuucguggaaugucagugccugg

SEQ ID NO: 15 - RNA, 38 nt, Artificial
  RNA aptamer
ggguacugguauauuggcgccuucguggaaugucagug

SEQ ID NO: 16 - RNA, 35 nt, Artificial
  RNA aptamer
ggguauauuggcgccucgggaaugucagugccugg

SEQ ID NO: 17 - RNA, 10 nt, Artificial
  consensus sequence of RNA aptamers
ggunayuggh

SEQ ID NO: 18 - RNA, 19 nt, Artificial
  partial sequence of RNA aptamer
ggcgccuucguggaauguc

SEQ ID NO: 19 - RNA, 80 nt, Artificial
  RNA aptamer
gggacgcucacguacgcucauuuuacuuuuccuacgaccgggugaacuggcucuuggaugucagugccuggacgugcagu

SEQ ID NO: 20 - RNA, 80 nt, Artificial
  RNA aptamer
gggacgcucacguacgcucaaaaugcuguugcagguuauuuggcucucggucugagaaugucagugccuggacgugcagu

SEQ ID NO: 21 - RNA, 80 nt, Artificial
  RNA aptamer
gggacgcucacguacgcucauguuccgggucgacuggcuguuagagaucucugauguaggucagugccuggacgugcagu

SEQ ID NO: 22 - RNA, 80 nt, Artificial
  RNA aptamer
gggacgcucacguacgcucagcuccggguauacuggcgacgaccguuauugugucgcaugucagugccuggacgugcagu

SEQ ID NO: 23 - RNA, 80 nt, Artificial
  RNA aptamer
gggacgcucacguacgcucagguguacuggcacuacugaaauuucauuugaguaggucugucagugccuggacgugcagu

SEQ ID NO: 24 - RNA, 80 nt, Artificial
  RNA aptamer
gggacgcucacguacgcucaggugaacugguccgcauuuagcuuucuuauuugcggguauucagugccuggacgugcagu

SEQ ID NO: 25 - RNA, 80 nt, Artificial
  RNA aptamer
gggacgcucacguacgcucagguguauuggaugcuuuaagcaggucucugcuucagcaauucagugccuggacgugcagu

SEQ ID NO: 26 - RNA, 80 nt, Artificial
  RNA aptamer
gggacgcucacguacgcucauucgaccggguuauuggcugcucuccucugguuugugaugucagugccuggacgugcagu

SEQ ID NO: 27 - RNA, 80 nt, Artificial
  RNA aptamer
gggacgcucacguacgcucaacacuugcuuuuucuuguccggguuuauuggucguuguauucagugccuggacgugcagu

SEQ ID NO: 28 - RNA, 80 nt, Artificial
  RNA aptamer
gggacgcucacguacgcucagagaucguucugguuauuggcgccuucugauaaaggaaugucagugccuggacgugcagu

SEQ ID NO: 29 - RNA, 80 nt, Artificial
  RNA aptamer
gggacgcucacguacgcucauugucuugguguauugguuacuguccaaugggcgguguauucagugccuggacgugcagu

SEQ ID NO: 30 - RNA, 80 nt, Artificial; identical to SEQ ID NO: 20
  RNA aptamer

SEQ ID NO: 31 - RNA, 80 nt, Artificial
  RNA aptamer
gggacgcucacguacgcucacgguggauuggcgacgaugaccuugauaguccucguaaugucagugccuggacgugcagu

SEQ ID NO: 32 - RNA, 80 nt, Artificial
  RNA aptamer
gggacgcucacguacgcucauagaguguauuuguaccagguauacuggcgcgaacgaaugucagugccuggacgugcagu

SEQ ID NO: 33 - RNA, 80 nt, Artificial
  RNA aptamer
gggacgcucacguacgcucagcucucuuacuuccugggugacuggcucuuucgggguaugucagugccuggacgugcagu

SEQ ID NO: 34 - RNA, 80 nt, Artificial
  RNA aptamer
gggacgcucacguacgcucagguuauuggcgcccucgaaccaaaauggaugccgggaaugucagugccuggacgugcagu

SEQ ID NO: 35 - RNA, 80 nt, Artificial
  RNA aptamer
gggacgcucacguacgcucacauguccggguggauuggaucgauuacuuguuuucguuuaucagugccuggacgugcagu

SEQ ID NO: 36 - RNA, 80 nt, Artificial
  RNA aptamer
gggacgcucacguacgcucaccucaagucgggucuauugucuccggcgaagcauggacugucagugccuggacgugcagu

SEQ ID NO: 37 - RNA, 80 nt, Artificial
  RNA aptamer
gggacgcucacguacgcucagagccacggguuuacuggcgcuaaacaaauguuuaggaugucagugccuggacgugcagu

SEQ ID NO: 38 - RNA, 80 nt, Artificial
  RNA aptamer
gggacgcucacguacgcucagcgcuucucguuugcuuuccggguucauugguccauguuuucagugccuggacgugcagu

SEQ ID NO: 39 - RNA, 80 nt, Artificial
  RNA aptamer
gggacgcucacguacgcucaggcguucuucgcuguaguuccgguuuauuggucuuuguuuucagugccuggacgugcagu

SEQ ID NO: 40 - RNA, 80 nt, Artificial
  RNA aptamer
gggacgcucacguacgcucaugucucgguuuauuggcggucggacuuuugcccugcgaugucagugccuggacgugcagu

SEQ ID NO: 41 - RNA, 80 nt, Artificial
  RNA aptamer
gggacgcucacguacgcucacgaaauccagguuugauuggcguggcacccuugccaagugucagugccuggacgugcagu

SEQ ID NO: 42 - RNA, 80 nt, Artificial
  RNA aptamer
gggacgcucacguacgcucaaugagcucaccuggguaauuggcgccaauucaagggucugucagugccuggacgugcagu

SEQ ID NO: 43 - RNA, 80 nt, Artificial
  RNA aptamer
gggacgcucacguacgcucacgcucaggugaauugguuacguuuucucugacaauguggaucagugccuggacgugcagu

SEQ ID NO: 44 - RNA, 80 nt, Artificial
  RNA aptamer
gggacgcucacguacgcucaauucuguucugucucuccggguuuacuggcgcuaugaaugucagugccuggacgugcagu

SEQ ID NO: 45 - RNA, 80 nt, Artificial
  RNA aptamer
gggacgcucacguacgcucaaagugucugcaagucuaccgguuuauuggccacuccguuuucagugccuggacgugcagu

SEQ ID NO: 46 - RNA, 80 nt, Artificial
  RNA aptamer
gggacgcucacguacgcucaugauugaaugggcgaaucgaccuuaccgguuuucugcaacucagugccuggacgugcagu

SEQ ID NO: 47 - RNA, 79 nt, Artificial
  RNA aptamer
gggacgcucacguacgcucaucucgccgcauuuccagguuuuuuggcgcuuaugaaugaucagugccuggacgugcagu

SEQ ID NO: 48 - RNA, 80 nt, Artificial; identical to SEQ ID NO: 44
  RNA aptamer

SEQ ID NO: 49 - RNA, 77 nt, Artificial
  RNA aptamer
gggacgcucacguacgcucagguggacugguuucuaagugcuuugacugcuggaggaucagugccuggacgugcagu

SEQ ID NO: 50 - RNA, 66 nt, Artificial
  RNA aptamer
gggacgcucacguacgcucagguuauuggcuuuccgagcgaagaugucagugccuggacgugcagu

SEQ ID NO: 51 - RNA, 80 nt, Artificial
  RNA aptamer
gggacgcucacguacgcucagguguauuggauaacagcugcuucuuggaacguugucguuucagugccuggacgugcagu

SEQ ID NO: 52 - RNA, 80 nt, Artificial
  RNA aptamer
gggacgcucacguacgcucagguuuauuggauguuugucucccguucgggacauucguuuucagugccuggacgugcagu

SEQ ID NO: 53 - RNA, 80 nt, Artificial
  RNA aptamer
gggacgcucacguacgcucagguugaucccguucuucuugacuggcgccuucauggagugucagugccuggacgugcagu

SEQ ID NO: 54 - RNA, 58 nt, Artificial
  RNA aptamer
ggguacgcucagguuuauuggugccguguaguggaaugucagugccuggacgugcagu

SEQ ID NO: 55 - RNA, 42 nt, Artificial
  RNA aptamer
gggucagguauauuggcgccuucguggaaugucagugccugg

SEQ ID NO: 56 - RNA, 40 nt, Artificial
  RNA aptamer
gggucagguauauuggcgccucgggaaugucagugccugg

SEQ ID NO: 57 - DNA, 420 bp, Artificial
  polynucleotide
atgcggggttctcatcatcatcatcatcatggtatggctagcatgactggtggacagcaaatgggtcgggatctgtacgacgatgacgataaggatcgatggggatcccaggtgcagctacaagaatctgggggtggcctggtgcaggcgggcggttccctgcgtctctccgcggcagcctctggccgcaccttcagtagctatggcatgggctggtttcgtcaggctccgggcaaagaacgtgaattcgtcgcagcgatcagctggtctggcggttccacctactatgcagacagcgtgaaaggccgcttcaccatctcccgggacaacgcgaaaaacaccgtgtacctgcaaatgaacagtctgaaaccggaagacacggccgtttattacgctgcagcggtttccagcggccgctaa

SEQ ID NO: 58 - DNA, 24 bp, Artificial
  polynucleotide
accttcagtagctatggcatgggc

SEQ ID NO: 59 - DNA, 39 bp, Artificial
  polynucleotide
ttcgtcgcagcgatcagctggtctggcggttccacctac

SEQ ID NO: 60 - protein, 11 aa, Artificial
  polypeptide
MRGSHHHHHHG

SEQ ID NO: 61 - DNA, 33 bp, Artificial
  DNA encoding a tag region of tagged protein
atgcggggttctcatcatcatcatcatcatggt

SEQ ID NO: 62 - protein, 11 aa, Artificial
  polypeptide
MASMTGGGGMG

SEQ ID NO: 63 - DNA, 33 bp, Artificial
  DNA encoding a tag region of tagged protein
atggctagcatgactggtggacagcaaatgggt

SEQ ID NO: 64 - protein, 14 aa, Artificial
  polypeptide
RDLYDDDDKDRWGS

SEQ ID NO: 65 - RNA, 54 nt, Artificial
  RNA aptamer
ggguacgcucagguauauuggcgccccgggaaugucagugccuggacgugcagu

SEQ ID NO: 66 - RNA, 55 nt, Artificial
  RNA aptamer
ggguacgcucagguauauuggcgccuucguggaugucagugccuggacgugcagu

SEQ ID NO: 67 - RNA, 54 nt, Artificial
  RNA aptamer
ggguacgcucagguauauuggcgccuucguggugucagugccuggacgugcagu

SEQ ID NO: 68 - RNA, 52 nt, Artificial
  RNA aptamer
ggguacgcucagguauauuggcgccucgggugucagugccuggacgugcagu

SEQ ID NO: 69 - DNA, 550 bp, Artificial
  polynucleotide
catatgcggggttctcatcatcatcatcatcatggtatggctagcatgactggtggacagcaaatgggtcgggatctgtacgacgatgacgataaggatcgatggggatcccaggtgcagctacaagaatctgggggtggcctggtgcaggcgggcggttccctgcgtctctccgcggcagcctctggccgcaccttcagtagctatggcatgggctggtttcgtcaggctccgggcaaagaacgtgaattcgtcgcagcgatcagctggtctggcggttccacctactatgcagacagcgtgaaaggccgcttcaccatctcccgggacaacgcgaaaaacaccgtgtacctgcaaatgaacagtctgaaaccggaagacacggccgtttattacgctgcagcggtttccagcggccgctaatctagataggtaatctctgcttaaaagcacagaatctaagatccctgccaggtatattggcgccttcgtggaatgtcagtgcctggcggggatttttttatttgttttcaggaaataaataatcgat

SEQ ID NO: 70 - DNA, 622 bp, Artificial
  polynucleotide
gctagcggtgaggtatattggcgccttcgtggaatgtcagtgcctcaccgctagcgcatatccagtgtagtaaggcaagtcccttcaagagttatcgttgatacccctcgtagtgcacattcctttaacgcttcaaaatctgtaaagcacgccatatcgccgaaaggcacacttaattattaagaggtaataccatatgcggggttctcatcatcatcatcatcatggtatggctagcatgactggtggacagcaaatgggtcgggatctgtacgacgatgacgataaggatcgatggggatcccaggtgcagctacaagaatctgggggtggcctggtgcaggcgggcggttccctgcgtctctccgcggcagcctctggccgcaccttcagtagctatggcatgggctggtttcgtcaggctccgggcaaagaacgtgaattcgtcgcagcgatcagctggtctggcggttccacctactatgcagacagcgtgaaaggccgcttcaccatctcccgggacaacgcgaaaaacaccgtgtacctgcaaatgaacagtctgaaaccggaagacacggccgtttattacgctgcagcggtttccagcggccgctaatctaga

SEQ ID NO: 71 - DNA, 76 bp, Artificial
  oligonucleotide
gctacgctgcagcgvnkvnkvnkvnktatvnkvnkvnkvnkvnkvnkvnkvnktacgactactggggccagggcac

SEQ ID NO: 72 - DNA, 76 bp, Artificial
  oligonucleotide
gctacgctgcagcgvnkvnkvnkvnkvnkvnkvnkvnktatvnkvnkvnkvnktacgactactggggccagggcac

SEQ ID NO: 73 - DNA, 54 bp, Artificial
  oligonucleotide
cacttagcggccgctggaaacggtcacctgggtgccctggccccagtagtcgta

SEQ ID NO: 74 - DNA, 95 bp, Artificial
  oligonucleotide
cacttagcggccgctcacgtaggcttgctgcaagtcgatggtgcaactctaccgctggaaacggtaacctgagtgccctggccccagtagtcgta

SEQ ID NO: 75 - DNA, 26 bp, Artificial
  primer
ggctagcatgactggtggacagcaaa

SEQ ID NO: 76 - DNA, 20 bp, Artificial
  primer
ggcagggatcttagattctg

SEQ ID NO: 77 - DNA, 25 bp, Artificial
  primer
cggaagacacggccgtttattacgc

SEQ ID NO: 78 - DNA, 25 bp, Artificial
  primer
tctagattagcggccgctggaaacg

SEQ ID NO: 79 - DNA, 44 bp, Artificial
  primer
ctagtagcggccgcttatctaccgctggaaacggtcacctgggt

SEQ ID NO: 80 - DNA, 24 bp, Artificial
  primer
cacttagcggccgctcacgtaggc

SEQ ID NO: 81 - protein, 34 aa, Artificial
  polypeptide
YYAAARKQIRSRAYVRLDYDYWGQGTQVTVSSGR

SEQ ID NO: 82 - protein, 34 aa, Artificial
  polypeptide
YYAAAVVLVDRPVYRRKRYDYWGQGTQVTVSSGR

SEQ ID NO: 83 - protein, 34 aa, Artificial
  polypeptide
YYAAARIMSRKAQYVMDSYDYWGQGTQVTVSSGR

SEQ ID NO: 84 - protein, 34 aa, Artificial
  polypeptide
YYAAASGRRYTISRHLSKYDYWGQGTQVTVSSGR

SEQ ID NO: 85 - protein, 34 aa, Artificial
  polypeptide
YYAAAMIALYQRIMRLLIYDYWGQGTQVTVSSGR

SEQ ID NO: 86 - protein, 34 aa, Artificial
  polypeptide
YYAAARGMGYTRVVDTVSYDYWGQGTQVTVSSGR

SEQ ID NO: 87 - protein, 33 aa, Artificial
  polypeptide
YYAAAAGMAYLRLIRLMYDYWGQGTQVTVSSGR

SEQ ID NO: 88 - protein, 34 aa, Artificial
  polypeptide
YYAAAGITNDLDGYAQPQYDYWGQGTQVTVSSGR

SEQ ID NO: 89 - RNA, 40 nt, Artificial
  aptamer
ccggguuauuggcgcaauauugguauccuguauuggucug

SEQ ID NO: 90 - RNA, 40 nt, Artificial
  aptamer
cguccgaucgauacugguauauuggcgccuucguggaaug

SEQ ID NO: 91 - RNA, 40 nt, Artificial
  aptamer
ccuguuuugucuagguuuauuggcgcuuauuccuggaaug

SEQ ID NO: 92 - RNA, 40 nt, Artificial
  aptamer
cucaggugauuggcgcuauuuaucgaucgauaauugaaug

SEQ ID NO: 93 - RNA, 40 nt, Artificial
  aptamer
uguuccuuuggguuauuggcuccuuguugaccaggggaug

SEQ ID NO: 94 - RNA, 40 nt, Artificial
  aptamer
caacacucgaaggguuuauuggccccaccaugguggaaug

SEQ ID NO: 95 - RNA, 40 nt, Artificial
  aptamer
cgguuauuggcggaggaucugucauggcaugccucgacug

SEQ ID NO: 96 - RNA, 40 nt, Artificial
  aptamer
cuucuuucccacucacgucucgguuuuauugguccaguuu

SEQ ID NO: 97 - RNA, 40 nt, Artificial
  aptamer
ggugaauuggcacuucuuuaucuacggaucgagucggaug

SEQ ID NO: 98 - RNA, 27 nt, Artificial
  aptamer
gguuuauuggugccguguaguggaaua

SEQ ID NO: 99 - RNA, 40 nt, Artificial
  aptamer
cuucccuagacccuccagguuacaggcgccgcccggaaug

SEQ ID NO: 100 - RNA, 25 nt, Artificial
  aptamer
gguauauuggcgccuucguggaaug

SEQ ID NO: 101 - RNA, 23 nt, Artificial
  aptamer
gguauauuggcgccucgggaaug

SEQ ID NO: 102 - RNA, 25 nt, Artificial
  aptamer
gguauauuggcgccuucguggaaug

SEQ ID NO: 103 - RNA, 29 nt, Artificial
  aptamer
uacugguauauuggcgccuucguggaaug

SEQ ID NO: 104 - RNA, 23 nt, Artificial
  aptamer
gguauauuggcgccucgggaaug

SEQ ID NO: 105 - RNA, 40 nt, Artificial
  aptamer
uucgaccggguuauuggcugcucuccucugguuugugaug

SEQ ID NO: 106 - RNA, 40 nt, Artificial
  aptamer
acacuugcuuuuucuuguccggguuuauuggucguuguau

SEQ ID NO: 107 - RNA, 40 nt, Artificial
  aptamer
gagaucguucugguuauuggcgccuucugauaaaggaaug

SEQ ID NO: 108 - RNA, 40 nt, Artificial
  aptamer
uugucuugguguauugguuacuguccaaugggcgguguau

SEQ ID NO: 109 - RNA, 40 nt, Artificial
  aptamer
aaaugcuguugcagguuauuuggcucucggucugagaaug

SEQ ID NO: 110 - RNA, 40 nt, Artificial
  aptamer
cgguggauuggcgacgaugaccuugauaguccucguaaug

SEQ ID NO: 111 - RNA, 40 nt, Artificial
  aptamer
uagaguguauuuguaccagguauacuggcgcgaacgaaug

SEQ ID NO: 112 - RNA, 40 nt, Artificial
  aptamer
gcucucuuacuuccugggugacuggcucuuucgggguaug

SEQ ID NO: 113 - RNA, 40 nt, Artificial
  aptamer
gguuauuggcgcccucgaaccaaaauggaugccgggaaug

SEQ ID NO: 114 - RNA, 40 nt, Artificial
  aptamer
cauguccggguggauuggaucgauuacuuguuuucguuua

SEQ ID NO: 115 - RNA, 40 nt, Artificial
  aptamer
ccucaagucgggucuauugucuccggcgaagcauggacug

SEQ ID NO: 116 - RNA, 40 nt, Artificial
  aptamer
gagccacggguuuacuggcgcuaaacaaauguuuaggaug

SEQ ID NO: 117 - RNA, 40 nt, Artificial
  aptamer
gcgcuucucguuugcuuuccggguucauugguccauguuu

SEQ ID NO: 118 - RNA, 40 nt, Artificial
  aptamer
ggcguucuucgcuguaguuccgguuuauuggucuuuguuu

SEQ ID NO: 119 - RNA, 40 nt, Artificial
  aptamer
ugucucgguuuauuggcggucggacuuuugcccugcgaug

SEQ ID NO: 120 - RNA, 40 nt, Artificial
  aptamer
cgaaauccagguuugauuggcguggcacccuugccaagug

SEQ ID NO: 121 - RNA, 40 nt, Artificial
  aptamer
augagcucaccuggguaauuggcgccaauucaagggucug

SEQ ID NO: 122 - RNA, 40 nt, Artificial
  aptamer
cgcucaggugaauugguuacguuuucucugacaaugugga

SEQ ID NO: 123 - RNA, 40 nt, Artificial
  aptamer
auucuguucugucucuccggguuuacuggcgcuaugaaug

SEQ ID NO: 124 - RNA, 40 nt, Artificial
  aptamer
aagugucugcaagucuaccgguuuauuggccacuccguuu

SEQ ID NO: 125 - RNA, 40 nt, Artificial
  aptamer
ugauugaaugggcgaaucgaccuuaccgguuuucugcaac

SEQ ID NO: 126 - RNA, 39 nt, Artificial
  aptamer
ucucgccgcauuuccagguuuuuuggcgcuuaugaauga

SEQ ID NO: 127 - RNA, 23 nt, Artificial
  aptamer
gguauauuggcgccccgggaaug

SEQ ID NO: 128 - RNA, 24 nt, Artificial
  aptamer
gguauauuggcgccuucguggaug

SEQ ID NO: 129 - RNA, 23 nt, Artificial
  aptamer
gguauauuggcgccuucguggug

SEQ ID NO: 130 - RNA, 21 nt, Artificial
  aptamer
gguauauuggcgccucgggug

SEQ ID NO: 131 - RNA, 40 nt, Artificial
  aptamer
uuuuacuuuuccuacgaccgggugaacuggcucuuggaug

SEQ ID NO: 132 - RNA, 40 nt, Artificial; identical to SEQ ID NO: 109
  aptamer

SEQ ID NO: 133 - RNA, 40 nt, Artificial
  aptamer
uguuccgggucgacuggcuguuagagaucucugauguagg

SEQ ID NO: 134 - RNA, 40 nt, Artificial
  aptamer
gcuccggguauacuggcgacgaccguuauugugucgcaug

SEQ ID NO: 135 - RNA, 40 nt, Artificial
  aptamer
gguguacuggcacuacugaaauuucauuugaguaggucug

SEQ ID NO: 136 - RNA, 40 nt, Artificial
  aptamer
ggugaacugguccgcauuuagcuuucuuauuugcggguau

SEQ ID NO: 137 - RNA, 40 nt, Artificial
  aptamer
gguguauuggaugcuuuaagcaggucucugcuucagcaau

SEQ ID NO: 138 - RNA, 40 nt, Artificial; identical to SEQ ID NO: 123
  aptamer

SEQ ID NO: 139 - RNA, 37 nt, Artificial
  aptamer
gguggacugguuucuaagugcuuugacugcuggagga

SEQ ID NO: 140 - RNA, 26 nt, Artificial
  aptamer
gguuauuggcuuuccgagcgaagaug

SEQ ID NO: 141 - RNA, 40 nt, Artificial
  aptamer
gguguauuggauaacagcugcuucuuggaacguugucguu

SEQ ID NO: 142 - RNA, 40 nt, Artificial
  aptamer
gguuuauuggauguuugucucccguucgggacauucguuu

SEQ ID NO: 143 - RNA, 40 nt, Artificial
  aptamer
gguugaucccguucuucuugacuggcgccuucauggagug

SEQ ID NO: 144 - RNA, 27 nt, Artificial
  aptamer
gguuuauuggugccguguaguggaaug

SEQ ID NO: 145 - RNA, 25 nt, Artificial
  aptamer
gguauauuggcgccuucguggaaug

SEQ ID NO: 146 - RNA, 23 nt, Artificial
  aptamer
gguauauuggcgccucgggaaug

SEQ ID NO: 147 - RNA, 26 nt, Artificial
  aptamer
ggunayugghgccuucguggaauguc

SEQ ID NO: 148 - RNA, 27 nt, Artificial
  aptamer
gguauauuggcgccuucguggaauguc

SEQ ID NO: 149 - protein, 34 aa, Artificial
  polypeptide
YYAAAMRSSLKVSYMRIVYDYWGQGTQVTVSSGR

SEQ ID NO: 150 - protein, 34 aa, Artificial
  polypeptide
YYAAAQDRVYRDRTVALRYDYWGQGTQVTVSSGR

SEQ ID NO: 151 - protein, 34 aa, Artificial
  polypeptide
YYAAAIELGYLTKVGSLRYDYWGQGTQVTVSSGR

SEQ ID NO: 152 - protein, 34 aa, Artificial
  polypeptide
YYAAAVNQPYARRLVVTSYDYWGQGTQVTVSSGR

SEQ ID NO: 153 - protein, 34 aa, Artificial
  polypeptide
YYAAAKDNTTAVRYVVVMYDYWGQGTQVTVSSGR

SEQ ID NO: 154 - protein, 34 aa, Artificial
  polypeptide
YYAAALLRSYWSRIFGWYYDYWGQGTQVTVSSGR

SEQ ID NO: 155 - DNA, 42 bp, Artificial
  DNA encoding a tag region of tagged protein
cgggatctgtacgacgatgacgataaggatcgatggggatcc

SEQ ID NO: 156 - DNA, 476 bp, Artificial
  polynucleotide
catatgcggggttctcatcatcatcatcatcatggtatggctagcatgactggtggacagcaaatgggtcgggatctgtacgacgatgacgataaggatcgatggggatcccaggtgcagctacaagaatctgggggtggcctggtgcaggcgggcggttccctgcgtctctccgcggcagcctctggccgcaccttcagtagctatggcatgggctggtttcgtcaggctccgggcaaagaacgtgaattcgtcgcagcgatcagctggtctggcggttccacctactatgcagacagcgtgaaaggccgcttcaccatctcccgggacaacgcgaaaaacaccgtgtacctgcaaatgaacagtctgaaaccggaagacacggccgtttattacgctgcagcggtttccagcggccgctaagtggtgaggtatattggcgccttcgtggaatgtcagtgcctcaccattctaga

SEQ ID NO: 157 - RNA, 33 nt, Artificial
  aptamer
gguauauuggcgccuucguggaaugucagugcc

SEQ ID NO: 158 - DNA, 33 bp, Artificial
  polynucleotide
ggtatattggcgccttcgtggaatgtcagtgcc

(19) United States
(12) Patent Application Publication
(10) Pub. No.: US 2014/0227724 A1
(43) Pub. Date: Aug. 14, 2014

(54) NUCLEIC ACID CONSTRUCT FOR USE IN SCREENING FOR PEPTIDE ANTIBODY, AND SCREENING METHOD USING SAME

(71) Applicants: Shotaro Tsuji, Tokyo (JP); Makiko Tsuji, Tokyo (JP)

(72) Inventors: Shotaro Tsuji, Tokyo (JP); Makiko Tsuji, Tokyo (JP)

(21) Appl. No.: 14/346,221

(22) PCT Filed: Aug. 10, 2012
(86) PCT No.: PCT/JP2012/070475

§ 371 Date: Mar. 20, 2014

(30) Foreign Application Priority Data
Sep. 29, 2011 (JP) 2011-215049

(51) Int. Cl. G01N 33/53

(52) U.S. Cl. CPC G01N 33/5308
USPC 435/7.92; 435/320.1

(57) ABSTRACT

The present invention provides a novel tool for simply screening a candidate molecule for an antibody and a method for screening a candidate molecule for an antibody using the tool. A nucleic acid construct includes: (x) an encoding nucleic acid of an antibody candidate, obtained by inserting an encoding nucleic acid of any peptide into an encoding nucleic acid of an antibody; (y) an encoding nucleic acid of a peptide tag; and (z) an encoding nucleic acid of an aptamer that is bindable to the peptide tag are used. When this nucleic acid construct is expressed, a complex of a fusion transcript of the encoding nucleic acids (x) to (z) and a fusion translation product of the encoding nucleic acids (x) and (y) is formed. When this complex and an antigen are brought into contact with each other, and the complex binding to the antigen is recovered, peptide that is bindable to the antigen and the encoding nucleic acid of the peptide can be identified from a transcript of an encoding nucleic acid of the any peptide in the complex.

### TECHNICAL FIELD

The present invention relates to a nucleic acid construct for use in screening for peptide antibody and a screening method using the same.

### BACKGROUND ART

Binding molecules that specifically recognize targets and bind thereto are used widely, for example, in medical fields such as examinations, diagnoses, and treatments and are very important tools in the analysis of a disease and a disease state. Among them, the monoclonal antibodies are researched most widely because they are favorable in specifity and affinity to targets and in stability and in terms of cost. However, in immunization to ordinary animals, it is difficult to form antibodies for low molecular antigens or antibodies for antigens that are stored across species at a high degree, and antibodies that are specific to targets are not always available. In fact, there is the problem that, in spite of the fact that effective markers related to diseases have been reported, the markers cannot be applied to diagnoses and treatments because there are no specific antibodies.

Hence, in these years, methods of artificially forming peptide antibodies in place of forming antibodies using animals have been reported. Specific examples of the methods include a phage display method, a liposome method, an in vitro virus method (an mRNA display method) using a puromycin probe, and a peptide array method (Non-Patent Documents 1, 2, and 3). In whichever method, with respect to targets whose antibodies cannot be obtained by immunization, peptide antibodies can be separated by artificial selection.

However, for example, these methods have problems such as use of special reagents and apparatus, efficiency, and cost. Among the above-mentioned methods, for example, a phage display method is relatively implemented. However, since the phage display method still requires technical know-how, the result is affected by the experience and knowledge of the experimenter, and it is not easy for everyone to perform the phage display method.

### PRIOR ART DOCUMENTS

### Non-Patent Document

[Non-Patent Document 1] Smith, G. P. et al., Science, Vol. 228: pp. 1315-1317, 1985

[Non-Patent Document 2] Matheakis, L. C. et al., Proc. Natl. Acad. Sci. USA, Vol. 91: pp. 9022-9026, 1994

[Non-Patent Document 3] Keefe, A. D. et al., Nature, Vol.410: pp. 715-718, 2001

### SUMMARY OF INVENTION

### Problem to be Solved by the Invention

Hence, the present invention is intended to provide a novel tool for simply screening a candidate molecule of an antibody and a screening method for screening a candidate molecule of an antibody using the tool.

### Means for Solving Problem

The nucleic acid construct according to the present invention is a nucleic acid construct for expressing an antibody candidate to an antigen, the nucleic acid construct including the following encoding nucleic acids (x) to (z): (x) an encoding nucleic acid of an antibody candidate, obtained by inserting an encoding nucleic acid of any peptide into an encoding nucleic acid of a variable region of an antibody; (y) an encoding nucleic acid of a peptide tag; and (z) an encoding nucleic acid of a nucleic acid molecule that binds to the peptide tag, wherein the encoding nucleic acids (x), (y), and (z) are bound with one another so that the encoding nucleic acids (x), (y), and (z) are transcribed as a fusion transcript, and the encoding nucleic acids (x) and (y) are translated as a fusion translation product.

The screening method according to the present invention is a method for screening for an antibody peptide that binds to an antigen or an encoding nucleic acid of the antibody peptide using the nucleic acid construct according to the present invention, the method including the following steps (A) to (C): (A) a step of expressing the nucleic acid construct to form a complex of a fusion transcript obtained by transcribing the encoding nucleic acid of an antibody candidate (x), the encoding nucleic acid of a peptide tag (y), and the encoding nucleic acid of a nucleic acid molecule (z) and a fusion translation product obtained by translating the encoding nucleic acid of an antibody candidate (x) and the encoding nucleic acid of a peptide tag (y); (B) a step of bringing the complex and an antigen into contact with each other; and (C) a step of recovering the complex binding to the antigen.

### Effects of the Invention

The nucleic acid construct of the present invention can form a complex of the fusion transcript and the fusion translation product utilizing binding between the nucleic acid molecule that binds to the peptide tag obtained by transcribing the encoding nucleic acid (z) and the peptide tag obtained by translating the encoding nucleic acid (y). In the complex, the fusion transcript includes a transcript of the encoding sequence of the any peptide, and the fusion translation product includes the any peptide. Therefore, when the complex is bound to the antigen, the antibody candidate binding to the antigen can be identified by identification of the transcript in the complex, for example. As described above, according to the present invention, the antibody candidate that is bindable to the antigen and the encoding nucleic acid of the antibody candidate can be easily identified by simply forming the complex and recovering the complex binding to the antigen. Moreover it is possible to construct a chimeric antibody, a humanized antibody, a human antibody, and the like from the information on the identified antibody candidate and the identified encoding nucleic acid of the antibody candidate, for example. Accordingly, the present invention provides a very useful tool and method for screening for a novel antibody to an antigen, for example, in medical fields.

### BRIEF DESCRIPTION OF DRAWINGS

FIGS. 1A to 1I are schematic views schematically showing the respective steps in an example of the screening method according to the present invention.

FIG. 2 shows schematic views representing the respective assumable secondary structures of various aptamers.

FIG. 3 is a schematic view showing an assumable secondary structure of an aptamer #47s.

FIG. 4 shows schematic views schematically representing the respective vectors for library in the examples of the present invention.

FIG. 5 is a schematic view schematically showing a configuration of VHH.

FIG. 6A is a schematic view showing a principle of ELISA in the examples of the present invention. FIG. 6B is a graph showing expression levels of fusion proteins in the examples of the present invention.

FIG. 7A is a schematic view showing a principle of a measurement of mRNA in the examples of the present invention. FIG. 7B is an electrophoresis photograph showing each amount of mRNA binding to each fusion protein in the examples of the present invention.

FIG. 8 is a graph showing amino acid sequences of peptides each exerting a binding property to human intelectin-1 and binding strengths thereof in the examples of the present invention.

### DESCRIPTION OF EMBODIMENTS

### First Nucleic Acid Construct

The first nucleic acid construct according to the present invention is, as mentioned above, a nucleic acid construct for expressing an antibody candidate to an antigen, the nucleic acid construct including the following encoding nucleic acids (x) to (z): (x) an encoding nucleic acid of an antibody candidate, obtained by inserting an encoding nucleic acid of any peptide into an encoding nucleic acid of a variable region of an antibody; (y) an encoding nucleic acid of a peptide tag; and (z) an encoding nucleic acid of a nucleic acid molecule that binds to the peptide tag, wherein the encoding nucleic acids (x), (y), and (z) are bound with one another so that the encoding nucleic acids (x), (y), and (z) are transcribed as a fusion transcript, and the encoding nucleic acids (x) and (y) are translated as a fusion translation product.

In the present invention an “antibody candidate” means a peptide candidate for determining whether or not a binding property to a target antigen is exerted. The first nucleic acid construct according to the present invention is, for example, also referred to as a nucleic acid construct for screening for peptide that is bindable to a target antigen or an encoding nucleic acid of the peptide.

In the present invention, hereinafter, the any peptide is referred to as a “random peptide”, the amino acid sequence of the any peptide is referred to as an “any peptide sequence” or a “random peptide sequence”, an encoding nucleic acid of the any peptide is referred to as an “any encoding nucleic acid” or a “random encoding nucleic acid, and the nucleic acid sequence of the any encoding nucleic acid is referred to as an “any encoding sequence” or a “random encoding sequence”. In the present invention, hereinafter, the peptide tag is referred to as a “tag”, the amino acid sequence of the tag is referred to as a “peptide tag sequence” or a “tag sequence”, the encoding nucleic acid of the tag is referred to as a “tag-encoding nucleic acid” or “peptide tag-encoding nucleic acid”, and the nucleic acid sequence of the tag-encoding nucleic acid is referred to as a “tag-encoding sequence” or a “peptide tag-encoding sequence”. In the present invention, hereinafter, the nucleic acid molecule that binds to the tag is referred to as an “aptamer” or a “tag aptamer”, the sequence of the aptamer is referred to as an “aptamer sequence” or a “tag aptamer sequence”, the encoding nucleic acid of the aptamer is referred to as an “aptamer-encoding nucleic acid” or a “tag aptamer-encoding nucleic acid”, the sequence of the aptamer-encoding nucleic acid is referred to as an “aptamer-encoding sequence” or a “tag aptamer-encoding sequence”.

In the present invention, an antisense strand refers to a strand of a double stranded nucleic acid, capable of being a template of transcription, and a sense strand refers to the other strand complementary to the antisense strand and does not serve as a template of transcription. Since the transcript is complementary to the antisense strand, it has the same sequence as the sense strand except that T is replaced by U. The “transcript” is, for example, RNA and specifically mRNA. The “translation product” is, for example, peptide and encompasses the meaning of protein. In the present invention, the “peptide” refers to the substance in which at least two amino acid residues are bound, for example. In the present invention, for example, the peptide encompasses the meaning of so-called polypeptide, and the polypeptide encompasses the meaning of so-called oligopeptide. Generally, the oligopeptide is peptide having about at the most 10 amino acid residues, for example. In the present invention, the 5′ side is also referred to as the upstream side and the 3′ side is also referred to as the downstream side.

The first nucleic acid construct according to the present invention may be a single strand or a double strand, for example, and the latter is preferable. Although the terms, the “sense strand” and the “antisense strand” are used in the description of the present invention, this does not limit the first nucleic acid construct according to the present invention to a double stranded-nucleic acid. These terms are used for making it clear whether a nucleic acid is described as an antisense strand that is to be used as a template of transcription or as its complementary strand (sense strand) when the sequences of the respective encoding nucleic acids, the positional relationship thereof, and the like are described, for example.

The first nucleic acid construct according to the present invention is, for example, preferably DNA, and each encoding nucleic acid includes a DNA sequence and is preferably composed of a DNA sequence, for example.

The first nucleic acid construct according to the present invention can transcribe the antibody candidate-encoding nucleic acid (x), the tag-encoding nucleic acid (y), and the aptamer-encoding nucleic acid (z) as a fusion transcript. The fusion transcript is, for example, a fusion transcript including mRNA obtained by transcribing the antibody candidate-encoding nucleic acid (x), mRNA obtained by transcribing the tag-encoding nucleic acid (y), and an RNA aptamer obtained by transcribing the aptamer-encoding nucleic acid (z), and these RNAs are linked with one another. Moreover, the first nucleic construct according to the present invention can translate the antibody candidate-encoding nucleic acid (x) and the tag-encoding nucleic acid (y) as a fusion translation product. The fusion translation product is, for example, a fusion translation product including the antibody candidate obtained by translating the antibody candidate-encoding nucleic acid (x) and the tag obtained by translating the tag-encoding nucleic acid (y), and these peptides are linked with each another.

In the first nucleic acid construct according to the present invention, the positional relationship between the antibody candidate-encoding nucleic acid (x), the tag-encoding nucleic acid (y), and the aptamer-encoding nucleic acid (z) is not particularly limited. As to the positional relationship between the antibody candidate-encoding nucleic acid (x) and the tag-encoding nucleic acid (y), for example, in the sense strand, the tag-encoding nucleic acid (y) may be positioned at the 5′ side of the antibody candidate-encoding nucleic acid (x) or at the 3′ side of the antibody candidate-encoding nucleic acid (x), and the former is preferable. As to the position of the aptamer-encoding nucleic acid (z), for example, in the sense strand, the aptamer-encoding nucleic acid (z) may be positioned at either of the 5′ side and the 3′ side of the antibody candidate-encoding nucleic acid (x) or the tag-encoding nucleic acid (y), and the latter is preferable.

The antibody candidate-encoding nucleic acid (x), the tag-encoding nucleic acid (y), and the aptamer-encoding nucleic acid (z) are, for example, preferably arranged in order of the tag-encoding nucleic acid (y), the antibody candidate-encoding nucleic acid (x), and the aptamer-encoding nucleic acid (z) in the sense strand. The direction of arranging these sequences is not particularly limited, and for example, the tag-encoding nucleic acid (y), the antibody candidate-encoding nucleic acid (x), and the aptamer-encoding nucleic acid (z) may be arranged from the 5′ side toward the 3′ side or from 3′ side toward the 5′ side in the sense strand in this order. The tag-encoding nucleic acid (y), the antibody candidate-encoding nucleic acid (x), and the aptamer-encoding nucleic acid (z) are preferably arranged from the 5′ side toward the 3′ side in the sense strand in this order. The aptamer-encoding nucleic acid (z) is preferably included in a stem-loop structure at a transcription termination site or in the vicinity of the transcription termination site in the nucleic acid construct, for example.

In the antibody candidate-encoding nucleic acid (x), as mentioned above, the any encoding nucleic acid is inserted into an encoding nucleic acid of a variable region of an antibody (hereinafter also referred to as a “variable region-encoding nucleic acid”). The antibody candidate-encoding nucleic acid (x) may include only the variable region-encoding nucleic acid, into which the any encoding nucleic acid has been inserted or an encoding nucleic acid of the entire antibody including the variable region or a partial fragment of the antibody, for example.

In the present invention, a form of inserting the any encoding nucleic acid is not particularly limited. In the present invention, the insertion of the any encoding sequence encompasses the meanings of an insertion by adding the any encoding sequence to the end of the variable region-encoding nucleic acid, an insertion by adding the any encoding sequence to the inside of the variable region-encoding nucleic acid, and an insertion by substitution of the any encoding sequence for a partial region of the variable region-encoding nucleic acid, for example. Hereinafter, for the sake of convenience, in some cases, descriptions are provided with referring to the insertion by adding the any encoding sequence to the end of the variable region-encoding nucleic acid as “addition”, referring to the insertion by adding the any encoding sequence to the inside of the variable region-encoding nucleic acid as “insertion”, and referring to the insertion by substitution of the any encoding sequence for a partial region of the variable region-encoding nucleic acid as “substitution”. In the present invention, the “insertion of any encoding nucleic acid” may be any of them.

The any encoding nucleic acid may be added to the end of the variable region-encoding nucleic acid and/or inserted into the inside of the variable region-encoding nucleic acid without partially deleting the variable region-encoding nucleic acid, for example. The any encoding nucleic acid may be inserted into a deleted site obtained by deleting at least a partial region of the variable region-encoding nucleic acid, for example. In this case, for example, it can also be said that the any encoding nucleic acid is inserted by substitution of the any encoding nucleic acid for at least a partial region of the variable region-encoding nucleic acid in the antibody candidate-encoding nucleic acid. In the present invention, the insertion of the any encoding nucleic acid is not particularly limited and is preferably an insertion by the substitution, for example.

The kind of an antibody from which the variable region is derived, e.g., Ig is not at all limited. Examples of the Ig include a monomer, a dimer, and a pentamer. Examples of the subtype of the Ig include IgA, IgM, IgG, IgD, and IgE. The species from which the Ig is derived also is not particularly limited, and examples thereof include mammals such as human, Muridae such as a mouse and a guinea pig, a rabbit, and Camelidae such as a camel and a llama. The variable region may be, for example, a sequence designed artificially.

The variable region is not particularly limited, and examples thereof include VH domain, a VL domain, and a VHH domain. The variable region may be, for example, a combination of these domains. Specific examples of the variable region include scFv/sFv obtained by linking a VH domain and a VL domain by a linker peptide or the like and peptide obtained by coexpressing a VH domain and a VL domain or VpreB.

The variable region is, for example, particularly preferably a VHH domain. The VHH domain is, for example, a variable region of the Ig derived from Camelidae, and the Ig is a single-stranded heavy-chain antibody with a deletion of light chain. The VHH domain can prevent formation of a disulfide bond, for example, and thus, it is preferred that the VHH domain is a sequence in which a Cys residue is substituted by an amino acid residue other than a Cys residue, for example.

A site of the variable region, into which the any encoding nucleic acid is inserted, is not particularly limited and is, for example, preferably a hypervariable region that is to be a recognition region of the antigen. When the any encoding nucleic acid is added as mentioned above, a site to which the any encoding nucleic acid is added can be, for example, the end of the variable region. When the any encoding nucleic acid is inserted as mentioned above, a site into which the any encoding nucleic acid is inserted can be, for example, the inside of the variable region. When the any encoding nucleic acid is inserted by substitution as mentioned above, the site to be substituted may be, for example, the entire variable region or a partial region of the variable region. That is, for example, the entire variable region or a partial region of the variable region may be substituted by the any encoding nucleic acid, for example.

When the variable region is a VHH domain, specific examples of the site into which the any encoding nucleic acid is inserted (site to be subjected to addition, insertion and/or substitution) include a CDR1 region, a CDR2 region, and a CDR3 region, and the site may be any one of the regions, two of the regions, or all of the three regions. Among the regions, the site into which the any encoding nucleic acid is inserted is, for example, preferably the CDR3 region, and particularly preferably, the any encoding nucleic acid is inserted into the entire CDR3 region or a partial region of the CDR3 region. The any encoding nucleic acid may be, for example, subjected to any of addition, insertion, and substitution, and specifically, for example, it is preferred that the any encoding nucleic acid is inserted by substitution of the any encoding nucleic acid for the entire CDR3 region or a partial region of the CDR3 region.

The sequence of the any encoding nucleic acid is not at all limited. The any encoding nucleic acid may be, for example, abuse sequence randomly designed or abuse sequence designed on the basis of any amino acid sequence. The length of the any encoding nucleic acid is not particularly limited. In the case where the any encoding nucleic acid is inserted into the variable region-encoding nucleic acid by substitution, the sequence of the any encoding nucleic acid can be designed as appropriate according to the length of the deleted site in the variable region-encoding nucleic acid, for example. The length of the any encoding nucleic acid is, for example, a base length of any of multiples of 3. The lower limit of the length is not particularly limited and is, for example, 3-mer, preferably 12-mer, more preferably 18-mer, yet more preferably 36-mer. The upper limit of the length is not particularly limited and is, for example, 60-mer, preferably 51-mer, more preferably 45-mer. The range of the length is, for example, from 3-mer to 60-mer, preferably from 12-mer to 51-mer, more preferably from 36-mer to 45-mer.

The base sequence of the any encoding nucleic acid is preferably designed so that the frequency of stop codon becomes low in the middle of the sequence, for example. Therefore, for example, in the base sequence of the any encoding nucleic acid in a sense strand, the 3^{rd} base of the codon is preferably a base other than A, and specifically, the sequence of the codon is preferably “NNK”. Here, N is A, G, C, T, or U and K is G, T, or U. Further, in order to prevent the appearance of the stop codon, for example, in the base sequence of the any encoding nucleic acid in a sense strand, the 1^{st} base of the codon can be abase other than T, and specifically, the sequence of the codon can be “VNK”. Here, V is A, G, or C.

The any encoding nucleic acid is preferably arranged so as to forma reading frame according to the amino acid sequence of the any peptide, for example. Moreover, the any encoding nucleic acid is preferably inserted so as not to change a reading frame of the variable region-encoding nucleic acid or so as not to change a reading frame of the any encoding nucleic acid, for example.

In the first nucleic acid construct according to the present invention, each of the antibody candidate-encoding nucleic acid (x) and the tag-encoding nucleic acid (y) may include a start codon, for example, and either one of the encoding nucleic acids positioned at the 5′ side preferably includes a start codon. That is, when the antibody candidate-encoding nucleic acid. (x) is arranged at the 5′ side of the tag-encoding nucleic acid (y), the antibody candidate-encoding nucleic acid (x) preferably includes a start codon at the 5′ side thereof, for example. Moreover, in the first nucleic acid construct according to the present invention, when the tag-encoding nucleic acid (y) is arranged at the 5′ side of the antibody candidate-encoding nucleic acid (x), the tag-encoding nucleic acid (y) preferably includes a start codon at the 5′ side thereof. In the first nucleic acid construct according to the present invention, the latter is preferable.

When the antibody candidate-encoding nucleic acid (x), the tag-encoding nucleic acid (y), and the aptamer-encoding nucleic acid (z) are arranged from the 5′ side toward the 3′ side in this order, it is preferred that the antibody candidate-encoding nucleic acid (x) does not include a stop codon, and the tag-encoding nucleic acid (y) includes a stop codon at the 3′ side thereof. Specifically, in the tag-encoding nucleic acid (y), a stop codon is preferably adjacent to the 3′ side of a codon to a C-terminal amino acid residue of the tag. When the tag-encoding nucleic acid (y), the antibody candidate-encoding nucleic acid (x), and the aptamer-encoding nucleic acid (z) are arranged from the 5′ side toward the 3′ side in this order, it is preferred that the tag-encoding nucleic acid (y) does not include a stop codon, and the antibody candidate-encoding nucleic acid (x) includes a stop codon at the 3′ side thereof, for example. Specifically, in the antibody candidate-encoding nucleic acid (x), a stop codon is preferably adjacent to the 3′ side of a codon to a C-terminal amino acid residue of the antibody candidate. In the first nucleic acid construct according to the present invention, the latter is preferable. As described above, when the antibody candidate-encoding nucleic acid (x) arranged at the 5′ side than the aptamer-encoding nucleic acid (z) includes a stop codon, a translation of the aptamer-encoding nucleic acid can be sufficiently prevented, for example.

In the present invention, the kind of the tag and the kind of the nucleic acid molecule (aptamer) that binds to a peptide tag are not particularly limited as long as the peptide tag and the aptamer can be bound to each other. By selecting the tag and the aptamer bindable thereto, when the fusion transcript and the fusion translation product are formed by the first nucleic acid construct according to the present invention, the complex can be formed by the binding between the tag in the fusion translation product and the aptamer in the fusion transcript. In the present invention, the “tag” refers to peptide that is to be bound or added to a molecule as a marker, for example.

In the present invention, “bindable to a tag” can also be described as “having a binding capacity to a tag” or “having a binding activity to a tag (tag binding activity)”, for example. The binding between the aptamer and the tag can be determined, for example, by the surface plasmon resonance-molecular interaction analysis or the like. An apparatus such as Biacore X (product name, GE Healthcare UK Ltd.) can be used for the determination, for example. The binding activity of the aptamer to the tag can be expressed, for example, by the dissociation constant between the aptamer and the tag. In the present invention, the dissociation constant of the aptamer is not particularly limited.

For example, it is preferred that the aptamer is specifically bindable to the tag and it is more preferred that the aptamer has a superior binding force to the tag. The binding constant (K_{D}) of the aptamer to the tag is preferably 1×10 mol/L or less, more preferably 5×10⁻¹⁰ mol/L, and yet more preferably 1×10⁻¹⁰ mol/L, for example.

The aptamer is bindable to a single tag. In addition, the aptamer is bindable to a fusion peptide that includes the tag via the tag, for example. Examples of the fusion peptide include a fusion peptide that includes the tag at the N-terminal side, a fusion peptide that includes the tag at the C-terminal side, and a fusion peptide that includes the tag inside. Further, the fusion peptide may include other peptide. The length of the aptamer is not particularly limited and is, for example, from 20-mer to 160-mer, preferably from 30-mer to 120-mer, more preferably from 40-mer to 100-mer.

Examples of the tag include a histidine tag, a FLAG tag, an Xpress tag, a GST tag, and an antibody Fc region tag. Among them, the histidine tag is preferable. The length of the tag is not particularly limited, and the number of ammo acid residues is, for example, preferably from 6 to 330, from 6 to 33, or from 6 to 30, more preferably from 6 to 15, and yet more preferably from 8 to 15.

In the first nucleic acid construct according to the present invention, the tag-encoding nucleic acid (y) is arranged so as to form a reading frame according to the amino acid sequence of the tag, for example. Further, in the first nucleic acid construct according to the present invention, the tag-encoding nucleic acid (y) and the antibody candidate-encoding nucleic acid (x) are arranged so that the tag is added to the antibody candidate at the time of translation, for example. At the time of translation, for example, the tag may be added directly to the antibody candidate or added indirectly to the antibody candidate via a linker such as peptide.

In the present invention, hereinafter, histidine is also referred to as a “His”, a histidine tag is also referred to as a “His tag”, and an encoding nucleic acid of the His tag is also referred to as a “His tag-encoding nucleic acid”. Further, a nucleic acid molecule that is bindable to the His tag is also referred to as a “His tag aptamer” or an “aptamer”, and an encoding nucleic acid of the His tag aptamer is also referred to as a “His tag aptamer-encoding nucleic acid” or an “aptamer-encoding nucleic acid”.

The His tag normally means peptide having plural His, i.e., His peptide. In the present invention, for example, the His tag is peptide having plural contiguous His, and specifically, the His tag may be peptide composed of only plural continuous His or peptide including plural continuous His. In the latter case, for example, the peptide may further include an additional sequence at at least one of the N-terminal side and the C-terminal side of the plural continuous His. The additional sequence may be one amino acid residue or peptide composed of at least two amino acid residues, for example. In the first nucleic acid construct according to the present invention, the length of the His tag to be encoded with the His tag-encoding nucleic acid is not particularly limited. The number of amino acid residues of the His tag is, for example, from 6 to 30, preferably from 6 to 15, and more preferably from 8 to 15. The number of histidines in the His tag is, for example, preferably from 6 to 10, more preferably from 6 to 8, for example, and the number of continuous histidines is, for example, preferably from 6 to 10, more preferably from 6 to 8.

The sequence of the His tag-encoding nucleic acid is not particularly limited as long as the His tag-encoding nucleic acid includes a sequence that encodes a His peptide (hereinafter, referred to as a “His peptide-encoding sequence”). Specifically, for example, it is preferred that the sequence of the His tag-encoding nucleic acid has contiguous codons of His. Further, as mentioned above, the His tag may further include the additional sequence besides His peptide. Therefore, for example, the His tag-encoding nucleic acid may include a sequence that encodes the additional sequence (hereinafter, referred to as the “additional encoding sequence”) at at least one of the 5′ side and the 3′ side of the His peptide-encoding sequence. The additional encoding sequence is not particularly limited.

Specifically, for example, the additional encoding sequence at the 5′ side of the His peptide-encoding sequence can be a sequence including a start codon. The sequence including a start codon may include only the start codon or may include the start codon and a sequence having a base length of multiples of 3, for example. In the latter case, for example, the sequence having a base length of multiples of 3 is a sequence that encodes at least one amino acid residue and is adjacent to the 3′ side of the start codon, for example.

Further, the additional encoding sequence at the 3′ side of the His peptide-encoding sequence is preferably a sequence having a base length of multiples of 3, for example. Among them, in the case where the antibody candidate-encoding nucleic acid (x) is positioned at the 5′ side of a His tag-encoding nucleic acid (y), the additional encoding sequence at the 3′ side preferably includes a stop codon, for example. Specifically, for example, in the case where the antibody candidate-encoding nucleic acid (x), the His tag-encoding nucleic acid (y), and the aptamer-encoding nucleic acid (z) are arranged from the 5′ side toward the 3′ side in a sense strand in this order, the His tag-encoding nucleic acid (y) preferably includes a stop codon as mentioned above. On the other hand, in the case where the antibody candidate-encoding nucleic acid (x) is positioned at the 3′ side of a His tag-encoding nucleic acid (y), the additional encoding sequence at the 3′ side preferably does not include a stop codon. Specifically, for example, in the case where the His tag-encoding nucleic acid (y), the antibody candidate-encoding nucleic acid (x), and the aptamer-encoding nucleic acid (z) are arranged from the 5′ side toward the 3′ side in the sense strand in this order, the additional encoding sequence at the 3′ side preferably does not include a stop codon for efficiently translating the antibody candidate-encoding nucleic acid.

In the first nucleic acid construct according to the present invention, the aptamer-encoding nucleic acid (z) is not particularly limited as long as it is a nucleic acid that encodes a nucleic acid molecule (aptamer) that is bindable to the tag. Specific examples of the aptamer to be encoded with the aptamer-encoding nucleic acid are described below.

The first nucleic acid construct according to the present invention may include at least two tag-encoding nucleic acids, for example. In this case, one of the tag-encoding nucleic acids is the above-mentioned encoding nucleic acid of the tag to which the aptamer is bindable. Hereinafter, this tag-encoding nucleic acid is referred to as a “main peptide tag-encoding nucleic acid” and a tag to be encoded with this encoding nucleic acid is referred to as a “main peptide tag”. Examples of the main peptide tag include the above-mentioned tags and examples of the main peptide tag-encoding nucleic acid include the above-mentioned encoding nucleic acids. Preferably, the main peptide tag is the His tag, and the main peptide tag-encoding nucleic acid is the His tag-encoding nucleic acid. In the first the nucleic acid construct according to the present invention, hereinafter, a tag-encoding nucleic acid other than the main peptide tag-encoding nucleic acid is referred to as a “sub peptide tag-encoding nucleic acid”, and a tag to be encoded with this encoding nucleic acid is referred to as a “sub peptide tag”. The sub peptide tag and the sub peptide tag-encoding nucleic acid are not particularly limited. The sub peptide tag can be, for example, a T7 gene 10 leader, and the sub peptide tag-encoding nucleic acid can be, for example, an encoding sequence of the T7 gene 10 leader. In the case where the first nucleic acid construct according to the present invention includes the main peptide tag-encoding sequence and the sub peptide tag-encoding sequence, for example, a complex of a fusion transcript having a base sequence that includes the main peptide tag-encoding nucleic acid, the sub peptide tag-encoding nucleic acid, the antibody candidate-encoding nucleic acid, and the aptamer-encoding nucleic acid and a fusion translation product that includes the main peptide tag, the sub peptide tag, and the antibody candidate is formed by the transcription and the translation of the first nucleic acid construct according to the present invention. The position of the sub peptide tag-encoding nucleic acid is not particularly limited. For example, the sub peptide tag-encoding nucleic acid is preferably adjacent to the main peptide tag-encoding nucleic acid in a sense strand. The sub peptide tag-encoding nucleic acid can be arranged either of the 5′ side and the 3′ side of the main peptide tag-encoding nucleic acid, and is more preferably arranged at the 3′ side of the main peptide tag-encoding nucleic acid.

Specifically, for example, besides the His tag-encoding nucleic acid as the main peptide tag-encoding nucleic acid, the first nucleic acid construct according to the present invention may further include the sub peptide tag-encoding nucleic acid. In this case, for example, a complex of a fusion transcript having a base sequence that includes the His tag-encoding nucleic acid, the sub peptide tag-encoding nucleic acid, the antibody candidate-encoding nucleic acid, and the aptamer-encoding nucleic acid and a fusion translation product that includes the His tag, the sub peptide tag, and the antibody candidate is formed by the transcription and the translation of the first nucleic acid construct according to the present invention. The sub peptide tag is, for example, preferably a T7 gene 10 leader, and the first nucleic acid construct according to the present invention preferably includes the encoding sequence of the T7 gene 10 leader as the sub peptide tag-encoding nucleic acid. The position of the sub peptide tag-encoding nucleic acid is not particularly limited. For example, the sub peptide tag-encoding nucleic acid is preferably adjacent to the His tag-encoding nucleic acid in a sense strand. The sub peptide tag-encoding nucleic acid can be arranged either of the 5′ side and the 3′ side of the His tag-encoding nucleic acid, and is more preferably arranged at the 3′ side of the His tag-encoding nucleic acid.

The first nucleic acid construct according to the present invention may further include a sequence that encodes a linker (hereinafter, referred to as a “linker-encoding sequence”), for example. The linker may be one amino acid residue or peptide composed of at least two amino acid residues, for example. The position of the linker-encoding sequence is not particularly limited, and examples thereof include a site between the peptide tag-encoding nucleic acid and the antibody candidate-encoding nucleic acid or the aptamer-encoding nucleic acid and a site between the antibody candidate-encoding nucleic acid and the aptamer-encoding nucleic acid.

When the transcription and the translation are performed using the first nucleic acid construct according to the present invention, as mentioned above, the transcript (RNA aptamer) of the aptamer-encoding nucleic acid generated by the transcription is desirably not translated into peptide based on its sequence information. Hence, preferably, the first nucleic acid construct according to the present invention further includes a sequence for preventing the translation of the aptamer, for example. The sequence for preventing the translation of the aptamer can be, for example, the above-mentioned stop codon. In the case where the aptamer-encoding nucleic acid is arranged at the 3′ side of the tag-encoding nucleic acid and the antibody candidate-encoding nucleic acid in a sense strand, the sequence for preventing the translation of the aptamer is preferably arranged between the above-mentioned encoding nucleic acids and the aptamer-encoding nucleic acid, for example.

The component of the first nucleic acid construct according to the present invention is not particularly limited, for example. The component is, for example, a nucleotide residue, Examples of the nucleotide residue include a deoxyribonucleotide residue and a ribonucleotide residue. The first nucleic acid construct according to the present invention may be composed of any of the deoxyribonucleotide residue and the ribonucleotide residue or includes both of them. The first nucleic acid construct according to the present invention is preferably DNA that includes or is composed of a deoxyribonucleotide residue, for example.

The first nucleic acid construct according to the present invention may include a modified nucleotide residue, for example. The modified nucleotide residue can be, for example, a modified sugar residue. Examples of the sugar residue include a ribose residue and a deoxyribose residue. A site to be modified in the sugar residue is not particularly limited and can be, for example, the 2′ position and/or the 4′ position of the sugar residue. Examples of the modification include methylation, fluorination, amination, and thiation. Examples of the modified nucleotide residue include 2′-methylpyrimidine residue and 2′-fluoropyrimidine, and specific examples thereof include 2′-methyluracil (2′-methylated-uracil nucleotide residue), 2′-Methyleytosine (2′-methylated-cytosine nucleotide residue), 2′-fluorouracil (2′-fluorinated-uracil nucleotide residue), 2′-fluorocytosine (2′-fluorinated-cytosine nucleotide residue), 2′-aminouracil (2′-aminated-uracil-nucleotide residue), 2′-amino cytosine (2′-aminated-cytosine nucleotide residue), 2′-thiouracil (2′-thiated-uracil nucleotide residue), and 2′-thiocytosine (2′-thiated-cytosine nucleotide residue).

The base in the nucleotide residue may be, for example, a natural base (non-artificial base) or a non-natural base (artificial base). Examples of the natural base include A, C, G, T, and U and modified bases thereof. Examples of the non-natural base include modified bases and altered bases, and the non-natural base preferably has the same function as in the natural base. Examples of the artificial base having the same function as in the natural base include an artificial base bindable to cytosine (c) in place of guanine (g), an artificial base bindable to guanine (g) in place of cytosine (c), an artificial base bindable to thymine (t) or uracil (u) in place of adenine (a), an artificial base bindable to adenine (a) in place of thymine (t), and an artificial base bindable to adenine (a) in place of uracil (u). Examples of the modified base include methylated bases, fluorinated bases, aminated based, and thiated bases. Specific examples of the modified base include 2′-methyluracil, 2′-mettylcytosine, 2′-fluorouracil, 2′-fluorocytosine, 2′-aminouracil, 2′-aminocytosine, 2′-thiouracil, and 2′-thiocytosine. In the present invention, for example, bases represented by a, g, c, t, and u encompass the meaning of the artificial base having the same function as in the natural base in addition to the meaning of the natural base.

The first nucleic acid construct according to the present invention may include an artificial nucleic acid monomer residue as a component, for example. Examples of the artificial nucleic acid monomer residue include PNA (peptide nucleic acid), LNA (Locked Nucleic Acid), and ENA (2′-O,4′-C-Ethylenebridged Nucleic Acid). Bases in the monomer residue may be the same as mentioned above, for example.

The first nucleic acid construct according to the present invention is preferably a vector, for example, Hereinafter, the vector is also referred to as an “expression vector”. The expression vector can be constructed, for example, by inserting the antibody candidate-encoding nucleic acid, the tag-encoding nucleic acid, and the aptamer-encoding nucleic acid into the basic skeleton of a vector. The basic skeleton of the vector is not particularly limited, and conventional vectors can be used, for example. Hereinafter, the basic skeleton of the vector is referred to as a “basic vector”. In the case where the basic vector includes the antibody candidate and the tag-encoding nucleic acid, for example, the expression vector can be constructed by inserting the aptamer-encoding nucleic acid into a desired site. Examples of the basic vector serving as the basic skeleton include a plasmid vector and a virus vector. Examples of the plasmid vector include Escherichia coli derived plasmid vectors such as pCold series (registered trademark, TAKARA BIO INC.), pET series (Merck, Invitrogen Corporation, etc.), pRSET series (Invitrogen Corporation), pBAD series (Invitrogen Corporation), pcDNA series (Invitrogen Corporation), pEF series (Invitrogen Corporation), pBR322, pBR325, pUC118, and pUC119; Bacillus subtilis derived plasmid vectors such as pUB110 and pTP5; and yeast derived plasmid vectors such as YEp13, YEp24, and YCp50. Further, examples of the virus vector include λ phage vectors such as Charon4A, Charon21A, EMBL3, EMBL4, λgt10, λgt11, and λZAP; filamentous phage vectors such as M13KE and pCANTAB5E; T7 phage vectors such as T7Select series; animal DNA virus vectors or RNA virus vectors such as retrovirus, vaccinia virus, and adenovirus; an insect virus vector such as baculovirus; and plant virus vectors. Among these basic vectors, for example, pCold which is a cold shock expression vector is preferable. Since such vectors can prevent the insolubilization of peptide expressed in a living cell such as Escherichia coli and can promote the solubilization thereof, the expressed peptide can be recovered without difficulty.

The first nucleic acid construct according to the present invention preferably includes a promoter such as a T7 promoter, a cold shock expression promoter (cspA promoter), a trp promoter, a lac promoter, a PL promoter, or a tac promoter so that the fusion translation product can be expressed efficiently, for example. Besides this, for example, the nucleic acid construct according to the present invention may include a terminator; a cis element such as an enhancer; a polyadenylation signal; a sequence of origin of replication (ori); a selection marker; a ribosome binding sequence such as an SD sequence or a KOZAK sequence; or a suppressor sequence. Examples of the selection marker include a dihydrofolate reductase gene, an ampicillin-resistant gene, and a neomycin-resistant gene.

In the first nucleic acid construct according to the present invention, as mentioned above, the sequence of the aptamer-encoding nucleic acid is satisfied as long as it is a nucleic acid that encodes the aptamer that is bindable to the tag. Although the His tag aptamer will be illustrated as the aptamer hereinafter, the present invention is not limited thereto.

The sequence of the His tag aptamer is not particularly limited as long as it is bindable to the His tag. The dissociation constant of the His tag aptamer is not particularly limited and is, for example, 1×10⁻⁹ mol/L or less. Since the dissociation constant (Kd) of an antibody to a His tag generally exceeds 1×10⁻⁹ noon, the His tag aptamer has a better binding affinity than the antibody. The dissociation constant of the His tag aptamer is preferably 5×10⁻¹⁰ mol/L or less, more preferably 1×10⁻¹⁰ mol/L or less. By utilizing such His tag aptamer, a complex of the fusion transcript and the fusion translation product can be formed really stably, for example.

The His tag aptamer is bindable to a single His tag. In addition, the His tag aptamer is bindable to a fusion peptide that includes the His tag via the His tag, for example. Examples of the fusion peptide include a fusion peptide that includes the His tag at the N-terminal side, a fusion peptide that includes the His tag at the C-terminal side, and a fusion peptide that includes the His tag inside. Further, the fusion peptide may further include other peptide fragment.

Specific examples of the His tag aptamer is described below. In the present invention, the aptamer is not limited to these examples. The His tag aptamer shown below is, for example, an aptamer having a dissociation constant of 1×10⁻⁹ mol/L or less and has a better binding affinity to the His tag than an ordinary antibody. The sequences shown below are the sequences of the His tag aptamer. For example, the sequence of the encoding nucleic acid of the His tag aptamer is a sequence having complementarity or identity to the sequence of the His tag aptamer below and is a sequence in which U is replaced with T.

For example, the His tag aptamer preferably includes any of the following polynucleotides (a), (b), (c), and (d):
(a) polynucleotide that includes the base sequence represented by SEQ ID NO: 17:

(SEQ ID NO: 17)
GGUNₙAYUₘGGH,

where, N represents A, G, C, U, or T, n or Nₙ represents the number of Ns, which is an integer from 1 to 3, Y represents U, T, C, m of Uₘ represents the number of Us, which is an integer from 1 to 3, and H represents U, T, C, or A;
(b) polynucleotide that includes a base sequence obtained by substitution, deletion, addition, and/or insertion of one or more bases in the base sequence of the polynucleotide (a) and is bindable to the His peptide;
(c) polynucleotide that includes the base sequence represented by SEQ ID NO: 18:

(SEQ ID NO: 18)
GGCGCCUUCGUGGAAUGUC;

and
(d) polynucleotide that includes a base sequence obtained by substitution, deletion, addition, and/or insertion of one or more bases in the base sequence of the polynucleotide (c) and is bindable to the His peptide.

Each of the polynucleotides (a) to (d) may be polynucleotide that includes or is composed of each base sequence. The His tag aptamer may be a nucleic acid that includes or is composed of any of the polynucleotides (a) to (d).

The polynucleotide (a) may be polynucleotide that is composed of or includes the base sequence of SEQ ID NO: 17. Hereinafter, the base sequence of SEQ ID NO: 17 is also referred to as a “binding motif sequence”. In the binding motif sequence of SEQ ID NO: 17, N represents A, G, C, U, T, in which A, G, C, or U is preferable, n of Nₙ represents the number of Ns, which is an integer from 1 to 3, Y represents U, or C, in which or C is preferable, m of Uₘ represents the number of Us, which is an integer from 1 to 3, and H represents U, T, C, or A, in which U, C, or A is preferable. The binding motif sequence is a consensus sequence that can be seen in the base sequences of SEQ ID NOs: 1 to 16 and the like described below. In the binding motif sequence, the number (n) of Ns in Nₙ is not particularly limited and is, for example, any of 1 (N), 2 (NN), and 3 (NNN), and Ns may be the same bases or different bases. In the binding motif sequence, the number (m) of Us in Uₘ is not particularly limited and is, for example, any of 1 (U), 2 (@U), and 3 (UUU).

In the polynucleotide (a), examples of the polynucleotide that includes the binding motif sequence include the following polynucleotides (a1) to (a4).

(a1) polynucleotide that includes a base sequence represented by any of SEQ ID NOs: 89 to 104.

In the polynucleotide (a1), the base sequence represented by each sequence number has the binding motif sequence. The polynucleotide (a1) may be, for example, polynucleotide that includes or is composed of the base sequence of the sequence number. The His tag aptamer may be, for example, nucleic acid that includes or is composed of the polynucleotide (a1). The base sequences represented by SEQ ID NOs: 89 to 104 are shown in Table 1 below. In Table 1, each underlined part represents the binding motif sequence of SEQ ID NO: 17. Hereinafter, each polynucleotide and each aptamer that includes polynucleotide in Table 1 may be indicated by Name shown on the left side of each sequence (the same applies hereinafter).

TABLE 1

| Name | Sequence | No. |
|---|---|---|
| #701 | CCGGGUUAUU GGCGCAAUAU UGGUAUCCUG UAUUGGUCUG | SEQ ID NO.: 89 |
| shot47 | CGUCCGAUCG AUACUGGUAU AUUGGCGCCU UCGUGGAAUG | SEQ ID NO.: 90 |
| #716 | CCUGUUUUGU CUAGGUUUAU UGGCGCUUAU UCCUGGAAUG | SEO ID NO.: 91 |
| #727 | CUCAGGUGAU UGGCGCUAUU UAUCGAUCGA UAAUUGAAUG | SEQ ID NO.: 92 |
| #704 | UGUUCCUUUG GGUUAUUGGC UCCUUGUUGA CCAGGGGAUG | SEQ ID NO.: 93 |
| #713 | CAACACUCGA AGGGUUUAUU GGCCCCACCA UGGUGGAAUG | SEQ ID NO.: 94 |
| #708 | CGGUUAUUGG CGGAGGAUCU GUCAUGGCAU GCCUCGACUG | SEQ ID NO.: 95 |
| #718 | CUUCUUUCCC ACUCACGUCU CGGUUUUAUU GGUCCAGUUU | SEQ ID NO.: 96 |
| #746 | GGUGAAUUGG CACUUCUUUA UCUACGGAUC GAGUCGGAUG | SEQ ID NO.: 97 |
| #714 | ---------- ---GGUUUAU UGGUGCCGUG UAGUGGAAUA | SEQ ID NO.: 98 |
| #733 | CUUCCCUAGA CCCUCCAGGU UACAGGCGCC GCCCGGAAUG | SEQ ID NO.: 99 |
| #47s | ---------- -----GGUAU AUUGGCGCCU UCGUGGAAUG | SEQ ID NO.: 100 |
| #47sT | ---------- -----GGUAU AUUGGCGCC- UCG-GGAAUG | SEQ ID NO.: 101 |
| shot47sss | ---------- -----GGUAU AUUGGCGCCU UCGUGGAAUG | SEQ ID NO.: 102 |
| #47M1 | ---------- -UACUGGUAU AUUGGCGCCU UCGUGGAAUG | SEQ ID NO.: 103 |
| #47sssT | ---------- -----GGUAU AUUGGCGCC- UCG GGAAUG | SEQ ID NO.: 104 |

The polynucleotide (a1) can be, for example, the following polynucleotide (a1-1): (a1-1) polynucleotide that includes abuse sequence represented by any of SEQ. ID NOs: 1 to 16.

In the polynucleotide (a1-1), the base sequence represented by each sequence number includes the base sequence of any of SEQ ID NOs: 89 to 104. The polynucleotide (a1-1) may be, for example, polynucleotide that includes or is composed of the base sequence of each sequence number. The His tag aptamer may be, for example, nucleic acid that includes or is composed of the polynucleotide (a1-1). The base sequences represented by SEQ ID NOs: 1 to 16 are shown in Table 2 below. In Table 2, each underlined part represents the binding motif sequence of SEQ. ID NO: 17. Hereinafter, each polynucleotide and each aptamer that includes the polynucleotide in Table 2 may be indicated by Name shown on the left side of each sequence (the same applies hereinafter).

TABLE 2

| | | | |
|---|---|---|---|
| Name | Sequence | SEQ ID NO.: | No. |

| | | |
|---|---|---|
| #701 | gggacgcuca cguacgcuca CCGGGUUAUU GGCGCAAUAU UGGUAUCCUG UAUUGGUCUG ucagugccug gacgugcagu | SEQ |
| | | ID |
| | | NO.: |
| | | 1 |
| shot47 | gggacgcuca cguacgcuca CGUCCGAUCG AUACUGGUAU AUUGGCGCCU UCGUGGAAUG ucagugccug gacgugcagu | SEQ |
| | | ID |
| | | NO.: |
| | | 2 |
| #716 | gggacgcuca cguacgcuca CCUGUUUUGU CUAGGUUUAU UGGCGCUUAU UCCUGGAAUG ucagugccug gacgugcagu | SEQ |
| | | ID |
| | | NO.: |
| #727 | gggacgcuca cguacgcuca CUCAGGUGAU UGGCGCUAUU UAUCGAUCGA UAAUUGAAUG ucagugccug gacgugcagu | SEQ |
| | | ID |
| | | NO.: |
| | | 4 |
| #704 | gggacgcuca cguacgcuca UGUUCCUUUG GGUUAUUGGC UCCUUGUUGA CCAGGGGAUG ucagugccug gacgugcagu | SEQ |
| | | ID |
| | | NO.: |
| | | 5 |
| #713 | gggacgcuca cguacgcuca CAACACUCGA AGGGUUUAUU GGCCCCACCA UGGUGGAAUG ucagugccug gacgugcagu | SEQ |
| | | ID |
| | | NO: |
| | | 6 |
| #708 | gggacgcuca cguacgcuca CGGUUAUUGG CGGAGGAUCU GUCAUGGCAU GCCUCGACUG ucagugccug gacgugcagu | SEQ |
| | | ID |
| | | NO.: |
| | | 7 |
| #718 | gggacgcuca cguacgcuca CUUCUUUCCC ACUCACGUCU CGGUUUUAUU GGUCCAGUUU ucagugccug gacgugcagu | SEQ |
| | | ID |
| | | NO.: |
| | | 8 |
| #746 | gggacgcuca cguacgcuca GGUGAAUUGG CACUUCUUUA UCUACGGAUC GAGUCGGAUG ucagugccug gacgugcagu | SEQ |
| | | ID |
| | | NO.: |
| | | 9 |
| #714 | gggacgcuca cguacgcuca ---------- ---GGUUUAU UGGUGCCGUG UAGUGGAAUA ucagugccug gacgugcagu | SEQ |
| | | ID |
| | | NO.: |
| | | 10 |
| #733 | gggacgcuca cguacgcuca CUUCCCUAGA CCCUCCAGGU UACAGGCGCC GCCCGGAAUG ucagugccug gacgugcagu | SEQ |
| | | ID |
| | | NO.: |
| | | 11 |
| #47s | 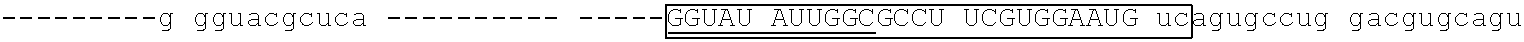 | SEQ ID |
| | | NO.: |
| | | 12 |
| #47sT | ---------g gguacgcuca ---------- -----GGUAU AUUGGCGCC- UCG-GGAAUG ucagugccug gacgugcagu | SEQ |
| | | ID |
| | | NO.: |
| | | 13 |
| shot47sss | ---------- ---------g ---------- -----GGUAU AUUGGCGCCU UCGUGGAAUG ucagugccug g | SEQ |
| | | ID |
| | | NO.: |
| | | 14 |
| #47M1 | ---------- -------ggg ---------- -UACUGGUAU AUUGGCGCCU UCGUGGAAUG ucagug | SEQ |
| | | ID |
| | | NO.: |
| | | 15 |
| #47sssT | ---------- ---------g ---------- -----GGUAU AUUGGCGCC- UCG GGAAUG ucagugccug g | SEQ |
| | | ID |
| | | NO.: |
| | | 16 |

(a2) polynucleotide that includes a base sequence represented by any of SEQ ID NOs: 105 to 114, 116 to 124, and 127 to 146.

In the polynucleotide (a2), the base sequence represented by each sequence number has the binding motif sequence. The polynucleotide (a2) may be, for example, polynucleotide that includes or is composed of the base sequence of the sequence number. The His tag aptamer may be, for example, nucleic acid that includes or is composed of the polynucleotide (a2). The base sequences represented by SEQ ID NOs: 105 to 114, 1.16 to 124, and 127 to 146 are shown in Tables 3 and 4 below. In Tables 3 and 4, each underlined part represents the binding motif sequence of SEQ ID NO: 17. Hereinafter, each polynucleotide and each aptamer that includes the polynucleotide in Tables 3 and 4 may be indicated by Name shown on the left side of each sequence (the same applies hereinafter).

TABLE 3

| Name | Sequence | No. |
|---|---|---|
| #730 | UUCGACCGGG UUAUUGGCUG CUCUCCUCUG GUUUGUGAUG | SEQ ID NO.: 105 |
| #743 | ACACUUGCUU UUUCUUGUCC GGGUUUAUUG GUCGUUGUAU | SEQ ID NO.: 106 |
| #7007 | GAGAUCGUUC UGGUUAUUGG CGCCUUCUGA UAAAGGAAUG | SEQ ID NO.: 107 |
| #7008 | UUGUCUUGGU GUAUUGGUUA CUGUCCAAUG GGCGGUGUAU | SEQ ID NO.: 108 |
| #7034 | AAAUGCUGUU GCAGGUUAUU UGGCUCUCGG UCUGAGAAUG | SEQ ID NO.: 109 |
| #707 | CGGUGGAUUG GCGACGAUGA CCUUGAUAGU CCUCGUAAUG | SEQ ID NO.: 110 |
| #715 | UAGAGUGUAU UUGUACCAGG UAUACUGGCG CGAACGAAUG | SEQ ID NO.: 111 |
| #719 | GCUCUCUUAC UUCCUGGGUG ACUGGCUCUU UCGGGGUAUG | SEQ ID NO.: 112 |
| #723 | GGUUAUUGGC GCCCUCGAAC CAAAAUGGAU GCCGGGAAUG | SEQ ID NO.: 113 |
| #725 | CAUGUCCGGG UGGAUUGGAU CGAUUACUUG UUUUCGUUUA | SEQ ID NO.: 114 |
| #736 | 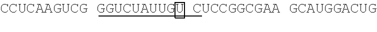 | SEQ ID NO.: 115 |
| #745 | GAGCCACGGG UUUACUGGCG CUAAACAAAU GUUUAGGAUG | SEQ ID NO.: 116 |
| #748 | GCGCUUCUCG UUUGCUUUCC GGGUUCAUUG GUCCAUGUUU | SEQ ID NO.: 117 |
| #7004 | GGCGUUCUUC GCUGUAGUUC CGGUUUAUUG GUCUUUGUUU | SEQ ID NO.: 118 |
| #7015 | UGUCUCGGUU UAUUGGCGGU CGGACUUUUG CCCUGCGAUG | SEQ ID NO.: 119 |
| #7029 | CGAAAUCCAG GUUUGAUUGG CGUGGCACCC UUGCCAAGUG | SEQ ID NO.: 120 |
| #7030 | AUGAGCUCAC CUGGGUAAUU GGCGCCAAUU CAAGGGUCUG | SEQ ID NO.: 121 |
| #7049 | CGCUCAGGUG AAUUGGUUAC GUUUUCUCUG ACAAUGUGGA | SEQ ID NO.: 122 |
| #7052 | AUUCUGUUCU GUCUCUCCGG GUUUACUGGC GCUAUGAAUG | SEQ ID NO.: 123 |
| #7054 | AAGUGUCUGC AAGUCUACCG GUUUAUUGGC CACUCCGUUU | SEQ ID NO.: 124 |
| #7009 | 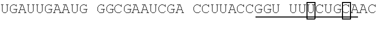 | SEQ ID NO.: 125 |
| #7062 | 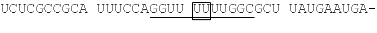 | SEQ ID NO.: 126 |
| #47sC3 | ---------- -----GGUAU AUUGGCGCC- CCG-GGAAUG | SEQ ID NO.: 127 |
| #47sA1 | ---------- -----GGUAU AUUGGCGCCU UCGUGGA-UG | SEQ ID NO.: 128 |
| #47sA | ---------- -----GGUAU AUUGGCGCCU UCGUGG--UG | SEQ ID NO.: 129 |
| #47sTA | ---------- -----GGUAU AUUGGCGCC- UCG-GG--UG | SEQ ID NO.: 130 |

TABLE 4

| Name | Sequence | No. |
|---|---|---|
| #627 | UUUUACUUUU CCUACGACCG GGUGAACUGG CUCUUCGAUG | SEQ ID NO.: 131 |
| #629 | AAAUGCUGUU GCAGGUUAUU UGGCUCUCGG UCUGAGAAUG | SEQ ID NO.: 132 |
| #504 | UGUUCCGGGU CGACUGGCUG UUAGAGAUCU CUGAUGUAGG | SEQ ID NO.: 133 |
| #505 | GCUCCGGGUA UACUGGCGAC GACCGUUAUU GUGUCGCAUG | SEQ ID NO.: 134 |
| #402 | GGUGUACUGG CACUACUGAA AUUUCAUUUG AGUAGGUCUG | SEQ ID NO.: 135 |
| #403 | GGUGAACUGG UCCGCAUUUA GCUUUCUUAU UUGCGGGUAU | SEQ ID NO.: 136 |
| #404 | GGUGUAUUGG AUGCUUUAAG CAGGUCUCUG CUUCAGCAAU | SEQ ID NO.: 137 |
| #405 | AUUCUGUUCU GUCUCUCCGG GUUUACUGGC GCUAUGAAUG | SEQ ID NO.: 138 |
| #303 | ---GGUGGAC UGGUUUCUAA GUGCUUUGAC UGCUGGAGGA | SEQ ID NO.: 139 |
| #304 | ---------- ----GGUUAU UGGCUUUCCG AGCGAAGAUG | SEQ ID NO.: 140 |
| #305 | GGUGUAUUGG AUAACAGCUG CUUCUUGGAA CGUUGUCGUU | SEQ ID NO.: 141 |
| #306 | GGUUUAUUGG AUGUUUGUCU CCCGUUCGGG ACAUUCGUUU | SEQ ID NO.: 142 |
| #AT5-5 | GGUUGAUCCC GUUCUUCUUG ACUGGCGCCU UCAUGGAGUG | SEQ ID NO.: 143 |
| #14sTT | ---------- ---GGUUUAU UGGUGCCGUG UAGUGGAAUG | SEQ ID NO.: 144 |
| #47ss | ---------- -----GGUAU AUUGGCGCCU UCGUGGAAUG | SEQ ID NO.: 145 |
| #47ssT | ---------- -----GGUAU AUUGGCGCC- UCG-GGAAUG | SEQ ID NO.: 146 |

The polynucleotide (a2) can be, for example, the following polynucleotide (a2-1): (a2-1)) polynucleotide that includes a base sequence represented by any of SEQ NOs: 26 to 35, 37 to 45, 65 to 68, 19 to 25, and 48 to 56.

In the polynucleotide (a2-1), the base sequences represented by SEQ NOs: 26 to 35, 37 to 45, 65 to 68, 19 to 25, and 48 to 56 includes the respective base sequences represented by SEQ ID NOs: 105 to 114, 116 to 124, and 127 to 146. The polynucleotide (a2-1) may be, for example, polynucleotide that includes or is composed of the base sequence of the sequence number. The His tag aptamer may be, for example, nucleic acid that includes or is composed of the polynucleotide (a2-1). The base sequences represented by SEQ ID NOs: 26 to 35, 37 to 45, 65 to 68, 19 to 25, and 48 to 56 are shown in Tables 5 and 6 below. In Tables 5 and 6, each underlined part represents the binding motif sequence of SEQ ID NO: 17. Hereinafter, each polynucleotide and each aptamer that includes the polynucleotide in Tables 5 and 6 may be indicated by Name shown on the left side of each sequence (the same applies hereinafter).

TABLE 5

| Name | Sequence | No. |
|---|---|---|
| #730 | gggacgcuca cguacgcuca UUCGACCGGG UUAUUGGCUG CUCUCCUCUG GUUUGUGAUG ucagugccug gacgugcagu | SEQ |
| | | ID |
| | | NO.: |
| | | 26 |
| #743 | gggacgcuca cguacgcuca ACACUUGCUU UUUCUUGUCC GGGUUUAUUG GUCGUUGUAU ucagugccug gacgugcagu | SEQ |
| | | ID |
| | | NO.: |
| | | 27 |
| #7007 | gggacgcuca cguacgcuca GAGAUCGUUC UGGUUAUUGG CGCCUUCUGA UAAAGGAAUG ucagugccug gacgugcagu | SEQ |
| | | ID |
| | | NO.: |
| | | 28 |
| #7008 | gggacgcuca cguacgcuca UUGUCUUGGU GUAUUGGUUA CUGUCCAAUG GGCGGUGUAU ucagugccug gacgugcagu | SEQ |
| | | ID |
| | | NO.: |
| | | 29 |
| #7034 | gggacgcuca cguacgcuca AAAUGCUGUU GCAGGUUAUU UGGCUCUCGG UCUGAGAAUG ucagugccug gacgugcagu | SEQ |
| | | ID |
| | | NO.: |
| | | 30 |
| #707 | gggacgcuca cguacgcuca CGGUGGAUUG GCGACGAUGA CCUUGAUAGU CCUCGUAAUG ucagugccug gacgugcagu | SEQ |
| | | ID |
| | | NO.: |
| | | 31 |
| #715 | gggacgcuca cguacgcuca UAGAGUGUAU UUGUACCAGG UAUACUGGCG CGAACGAAUG ucagugccug gacgugcagu | SEQ |
| | | ID |
| | | NO.: |
| | | 32 |
| #719 | gggacgcuca cguacgcuca GCUCUCUUAC UUCCUGGGUG ACUGGCUCUU UCGGGGUAUG ucagugccug gacgugcagu | SEQ |
| | | ID |
| | | NO.: |
| | | 33 |
| #723 | gggacgcuca cguacgcuca GGUUAUUGGC GCCCUCGAAC CAAAAUGGAU GCCGGGAAUG ucagugccug gacgugcagu | SEQ |
| | | ID |
| | | NO.: |
| | | 34 |
| #736 | 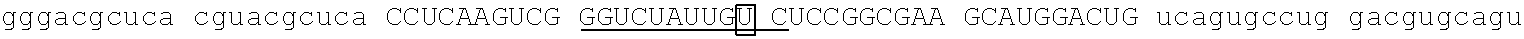 | SEQ ID |
| | | NO.: |
| | | 36 |
| #745 | gggacgcuca cguacgcuca GAGCCACGGG UUUACUGGCG CUAAACAAAU GUUUAGGAUG ucagugccug gacgugcagu | SEQ |
| | | ID |
| | | NO.: |
| | | 37 |
| #748 | gggacgcuca cguacgcuca GCGCUUCUCG UUUGCUUUCC GGGUUCAUUG GUCCAUGUUU ucagugccug gacgugcagu | SEQ |
| | | ID |
| | | NO.: |
| | | 38 |
| #7004 | gggacgcuca cguacgcuca GGCGUUCUUC GCUGUAGUUC CGGUUUAUUG GUCUUUGUUU ucagugccug gacgugcagu | SEQ |
| | | ID |
| | | NO.: |
| | | 39 |
| #7015 | gggacgcuca cguacgcuca UGUCUCGGUU UAUUGGCGGU CGGACUUUUG CCCUGCGAUG ucagugccug gacgugcagu | SEQ |
| | | ID |
| | | NO.: |
| | | 40 |
| #7029 | gggacgcuca cguacgcuca CGAAAUCCAG GUUUGAUUGG CGUGGCACCC UUGCCAAGUG ucagugccug gacgugcagu | SEQ |
| | | ID |
| | | NO.: |
| | | 41 |
| #7030 | gggacgcuca cguacgcuca AUGAGCUCAC CUGGGUAAUU GGCGCCAAUU CAAGGGUCUG ucagugccug gacgugcagu | SEQ |
| | | ID |
| | | NO.: |
| | | 42 |
| #7049 | gggacgcuca cguacgcuca CGCUCAGGUG AAUUGGUUAC GUUUUCUGUG ACAAUGUGGA ucagugccug gacgugcagu | SEQ |
| | | ID |
| | | NO.: |
| | | 43 |
| #7052 | gggacgcuca cguacgcuca AUUCUGUUCU GUCUCUCCGG GUUUACUGGC GCUAUGAAUG ucagugccug gacgugcagu | SEQ |
| | | ID |
| | | NO.: |
| | | 44 |
| #7054 | gggacgcuca cguacgcuca AAGUGUCUGC AAGUCUACCG GUUUAUUGGC CACUCCGUUU ucagugccug gacgugcagu | SEQ |
| | | ID |
| | | NO.: |
| | | 45 |
| #7009 | 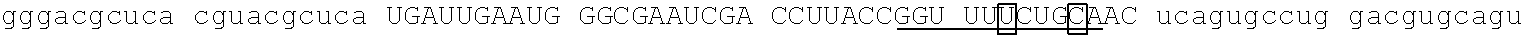 | SEQ ID |
| | | NO.: |
| | | 46 |
| #7062 | 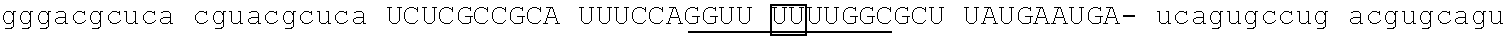 | SEQ ID |
| | | NO.: |
| | | 47 |
| #47sT | ---------g gguacgcuca ---------- -----GGUAU AUUGGCGCC- CCG-GGAAUG ucagugccug gacgug cagu | SEQ |
| | | ID |
| | | NO.: |
| | | 65 |
| #47sT | ---------g gguacgcuca ---------- -----GGUAU AUUGGCGCCU UCGUGGA-UG ucagugccug gacgu gcagu | SEQ |
| | | ID |
| | | NO.: |
| | | 66 |
| #47sA | ---------g gguacgcuca ---------- -----GGUAU AUUGGCGCCU UCGUGG--UG ucagugccug gacgug cagu | SEQ |
| | | ID |
| | | NO.: |
| | | 67 |
| #47sTA | ---------g gguacgcuca ---------- -----GGUAU AUUGGCGCC- UCG-GG--UG ucagugccug gacgugca gu | SEQ |
| | | ID |
| | | NO.: |
| | | 68 |

TABLE 6

| Name | Sequence | No. |
|---|---|---|
| #627 | gggacgcuca cguacgcuca UUUUACUUUU CCUACGACCG GGUGAACUGG CUCUUGGAUG ucagugccug gacgugcagu | SEQ ID |
| | | NO.: 19 |
| #629 | gggacgcuca cguacgcuca AAAUGCUGUU GCAGGUUAUU UGGCUCUCGG UCUGAGAAUG ucagugccug gacgugcagu | SEQ ID |
| | | NO.: 20 |
| #504 | gggacgcuca cguacgcuca UGUUCCGGGU CGACUGGCUG UUAGAGAUCU CUGAUGUAGG ucagugccug gacgugcagu | SEQ ID |
| | | NO.: 21 |
| #505 | gggacgcuca cguacgcuca GCUCCGGGUA UACUGGCGAC GACCGUUAUU GUGUCGCAUG ucagugccug gacgugcagu | SEQ ID |
| | | NO.: 22 |
| #402 | gggacgcuca cguacgcuca GGUGUACUGG CACUACUGAA AUUUCAUUUG AGUAGGUCUG ucagugccug gacgugcagu | SEQ ID |
| | | NO.: 23 |
| #403 | gggacgcuca cguacgcuca GGUGAACUGG UCCGAUUUA GCUUUCUUUAU UUGCGGGUAU ucagugccug gacgugcagu | SEQ ID |
| | | NO.: 24 |
| #404 | gggacgcuca cguacgcuca GGUGUAUUGG AUGCUUUAAG CAGGUCUCUG CUUCAGGAAU ucagugccug gacgugcagu | SEQ ID |
| | | NO.: 25 |
| #405 | gggacgcuca cguacgcuca AUUCUGUUCU GUCUCUCCGG GUUUACUGGC GCUAUGAAUG ucagugccug gacgugcagu | SEQ ID |
| | | NO.: 48 |
| #303 | gggacgcuca cguacgcuca ---GGUGGAC UGGUUUCUAA GUGCUUUGAC UGCUGGAGGA ucagugccug gacgugcagu | SEQ ID |
| | | NO.: 49 |
| #304 | gggacgcuca cguacgcuca ---------- ----GGUUAU UGGCUUUGCG AGCGAAGAUG ucagugccug gacgugcagu | SEQ ID |
| | | NO.: 50 |
| #305 | gggacgcuca cguacgcuca GGUGUAUUGG AUAACAGCUG CUUCUUGGAA CGUUGUCGUU ucagugccug gacgugcagu | SEQ ID |
| | | NO.: 51 |
| #306 | gggacgcuca cguacgcuca GGUUUAUUGG AUGUUUGUCU CCCGUUCGGG ACAUUCGUUU ucagugccug gacgugcagu | SEQ ID |
| | | NO.: 52 |
| #AT5-5 | gggacgcuca cguacgcuca GGUUGAUCCC GUUCUUCUUG ACUGGCGCCU UCAUGGAGUG ucagugccug gacgugcagu | SEQ ID |
| | | NO.: 53 |
| #14sTT | ---------g gguacgcuca ---------- ---GGUUUAU UGGUGCCGUG UAGUGGAAUG ucagugccug gacgugcagu | SEQ ID |
| | | NO.: 54 |
| #47ss | ---------- ----ggguca ---------- -----GGUAU AUUGGCGCCU UCGUGGAAUG ucagugccug g--------- | SEQ ID |
| | | NO.: 55 |
| #47ssT | ---------- ----ggguca ---------- -----GGUAU AUUGGCGCC- UCG-GGAAUG ucagugccug g--------- | SEQ ID |
| | | NO.: 56 |

(a3) polynucleotide that includes a base sequence represented by SEQ ID NO: 147:

(SEQ ID NO: 147)
GGUNnAYUmGGHGCCUUCGUGGAAUGUC.

In the base sequence of SEQ ID NO: 147, “GGUNₙAYUₘGGH” is the binding motif sequence of SEQ ID NO: 17. Further, in the base sequence of SEQ ID NO: 147, “GGHGCCUUCGUGGAAUGUC” is the base sequence represented by SEQ ID NO: 18 (where, H is C) described below. The base sequence of SEQ ID NO: 18 is, for example, the base sequence of a region that forms a stem-loop structure in an aptamer, and is hereinafter also referred to as a “stem-loop motif sequence”. In the base sequence of SEQ ID NO: 147, 3 bases at the 3′ end of the binding motif sequence overlap with 3 bases at the 5′ end of the stem-loop motif sequence.

The polynucleotide (a3) may be, for example, polynucleotide that includes or is composed of the base sequence of the sequence number. The His tag aptamer may be, for example, nucleic acid that includes or is composed of the polynucleotide (a3).

The base sequence of SEQ ID NO: 147 can be, for example, a base sequence represented by SEQ ID NO: 148: GGUAUAUUGGCGCCUUCGUGGAAUGUC (SEQ ID NO: 148).

The nucleic acid that includes the polynucleotide (a3) can be, for example, the following polynucleotide (a3-1): (a3-1) polynucleotide that includes a base sequence represented by any of SEQ ID NOs: 2, 12, 14, 15, and 55.

In the polynucleotide (a3-1), the base sequence represented by each sequence number includes the base sequence represented by SEQ ID NO: 147, specifically SEQ ID NO: 148. The polynucleotide (a3-1) may be, for example, polynucleotide that includes or is composed of the base sequence of the sequence number. The His tag aptamer may be, for example, nucleic acid that includes or is composed of the polynucleotide (a3-1). The base sequences represented by SEQ ID NOs: 2, 12, 14, 15, and 55 are shown in Table 7 below. In Table 7, each underlined part represents the base sequence of SEQ ID NO: 17, and each region enclosed in the box represents the base sequence of SEQ ID NO: 18. In addition, the His tag aptamer can be an aptamer represented by SEQ ID NO: 157, and the dissociation constant of this aptamer and a His tag is, for example, about 4×10⁻¹² M.

(SEQ ID NO: 157)
GGUAUAUUGGCGCCUUCGUGGAAUGUCAGUGCC

TABLE 7

| Name | Sequence | No. |
|---|---|---|
| shot47 | 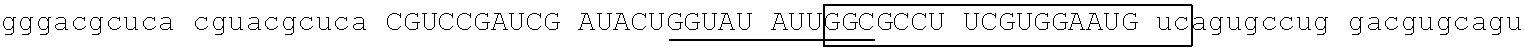 | SEQ ID NO.: 2 |
| #47s | 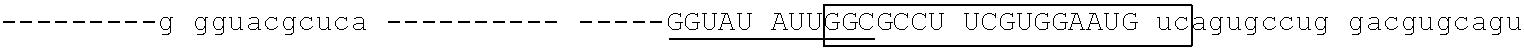 | SEQ ID NO.: 12 |
| #47sT | 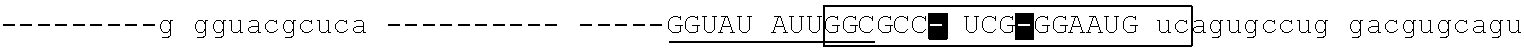 | SEQ ID NO.: 13 |
| #47sT | 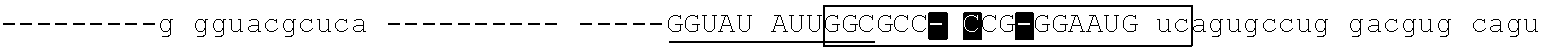 | SEQ ID NO.: 65 |
| #47sA1 | 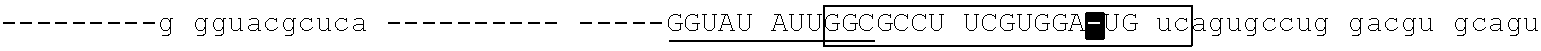 | SEQ ID NO.: 66 |
| #47sA | 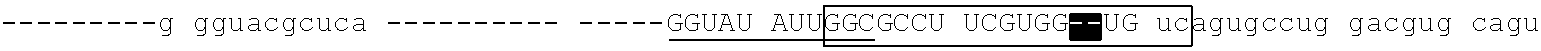 | SEQ ID NO.: 67 |
| #47sTA | 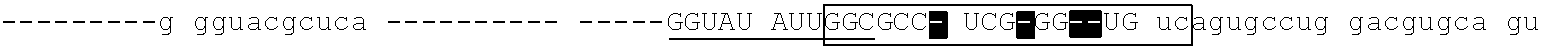 | SEQ ID NO.: 68 |
| shot47sss | 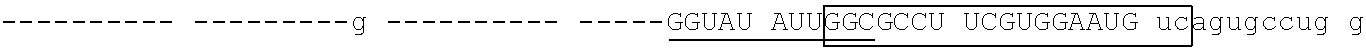 | SEQ ID NO.: 14 |
| #47M1 | 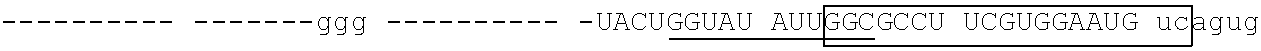 | SEQ ID NO.: 15 |
| #47sssT | 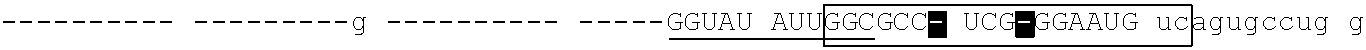 | SEQ ID NO.: 16 |
| #14sTT | 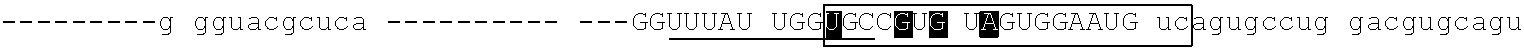 | SEQ ID NO.: 54 |
| #47ss | 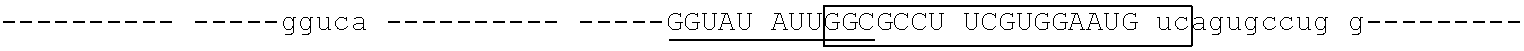 | SEQ ID NO.: 55 |
| #47ssT | 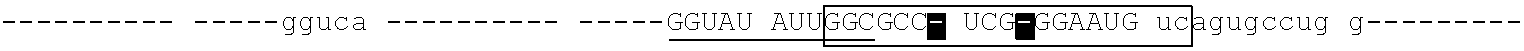 | SEQ ID NO.: 56 |

The polynucleotide (b) is, as mentioned above, polynucleotide that includes a base sequence obtained by substitution, deletion, addition, and/or insertion of one or more bases in the base sequence of the polynucleotide (a) and is bindable to the His peptide.

The polynucleotide (b) may be, for example, polynucleotide that is composed of or includes the base sequence. The His tag aptamer may be, for example, nucleic acid that includes or is composed of the polynucleotide (b).

In the polynucleotide (b), the “base sequence of the polynucleotide (a)” corresponds to any of the base sequences shown in the polynucleotides (a1) to (a3) besides the base sequence of SEQ ID NO: 17 mentioned above, for example (the same applies hereinafter).

In the polynucleotide (b), “one or more” is not particularly limited as long as the polynucleotide (b) is bindable to the His tag. The “one or more” is, for example, in the base sequence of SEQ ID NO: 17, from 1 to 5, preferably from 1 to 4, more preferably from 1 to 3, yet more preferably from 1 or 2, and particularly preferably 1. Further, the “one or more” is, for example, in the base sequence of each of the polynucleotides (a1) to (a3), from 1 to 10, preferably from 1 to 5, more preferably from 1 to 4, yet more preferably from 1 to 3, particularly preferably from 1 or 2, most preferably 1. The “one or more” is, for example, in the full-length base sequence of the aptamer that includes the polynucleotide (a), from 1 to 10, preferably from 1 to 5, more preferably from 1 to 4, yet more preferably from 1 to 3, particularly preferably 1 or 2, most preferably 1.

The base to be used in the substitution, addition, and/or insertion is not particularly limited, and examples thereof include the above-mentioned various bases. The substitution, addition, and/or insertion of the base may be performed by substitution, addition, and/or insertion of the nucleotide residue or the artificial nucleic acid monomer residue. The same applies hereinafter.

Examples of the polynucleotide (b) include the base sequences shown in Tables 3 and 5. Specific examples thereof include the base sequences represented by SEQ ID NOs: 115 (#736) and 36 (#736). The base sequence of SEQ ID NO: 36 includes the base sequence of SEQ ID NO: 115. Examples of the polynucleotide (b) further include the base sequences represented by SEQ ID NOs: 125 (#7009) and 46 (#7009). The base sequence of SEQ ID NO: 46 includes the base sequence of SEQ ID NO: 125. In Tables 3 and 5, each double underlined part of these base sequences corresponds to the binding motif sequence of SEQ ID NO: 17, and each base enclosed in the box is a substituted base which is different from the base sequence of SEQ ID NO: 17. Specific examples of the polynucleotide (b) further include the base sequences represented by SEQ ID NOs: 126 (#7062) and 47 (#7062). In Tables 3 and 5, each double underlined part of these base sequences corresponds to the binding motif sequence of SEQ ID NO: 17, and one of the bases (UU) enclosed in the box is a substituted base that is different from A of the binding motif sequence of SEQ ID NO: 17. Specific examples of the polynucleotide (b) further include the base sequences of SEQ NOs: 143 (#AT5-5) and 53 (#AT5-5),

The polynucleotide (c) is, as mentioned above, polynucleotide that includes the base sequence represented by SEQ ID NO: 18. The base sequence of SEQ ID NO: 18 is, for example, as mentioned above, the base sequence of a region that forms a stem-loop structure in aptamer.

(SEQ ID NO: 18)
GGCGCCUUCGUGGAAUGUC

The polynucleotide (c) may be, for example, polynucleotide that is composed of or includes the base sequence. The His tag aptamer may be, for example, nucleic acid that includes or is composed of the polynucleotide (c).

Examples of the polynucleotide (c) include base sequences represented by SEQ ID NOs: 2, 12, 14, 15, and 55. These base sequences are shown in Table 7,

The polynucleotide (d) is, as mentioned above, polynucleotide that includes a base sequence obtained by substitution, deletion, addition, and/or insertion of one or more bases in the base sequence of the polynucleotide (c) and is bindable to the His peptide.

The polynucleotide (d) may be polynucleotide that is composed of or includes the base sequence, The His tag aptamer may be, for example, nucleic acid that includes or is composed of the polynucleotide (d).

In the polynucleotide (d), “the base sequence of the polynucleotide (c)” corresponds to any of the listed base sequences of the sequence numbers besides the base sequence of SEQ ID NO: 18 mentioned above (the same applies hereinafter).

In the polynucleotide (d), “one or more” is not particularly limited as long as the polynucleotide (d) is bindable to the His tag. The “one or more” in the base sequence of SEQ ID NO: 18 is, for example, from 1 to 5, preferably from 1 to 4, more preferably from 1 to 3, yet more preferably from 1 or 2, particularly preferably 1. The “one or more” in the base sequence represented by each of SEQ. ID NOs: 2, 12, 14, 15, and 55 is, for example, from 1 to 10, preferably from 1 to 5, more preferably from 1 to 4, yet more preferably from 1 to 3, particularly preferably 1 or 2, most preferably 1. The “one or more” in the full-length base sequence of the aptamer that includes the polynucleotide (d) is, for example, from 1 to 10, preferably from 1 to 5, more preferably from 1 to 4, yet more preferably from 1 to 3, particularly preferably I or 2, most preferably 1. The polynucleotide (d) preferably has axiom-loop structure that is substantially the same as the stem-loop structure formed by the base sequence of SEQ ID NO: 18, for example.

Examples of the polynucleotide (d) include base sequences represented by SEQ NOs: 13, 65 to 68, 16, 54, and 56. These sequences are shown in Table 7. In the base sequences of SEQ NOs: 13, 65 to 68, 16, 54, and 56 shown in Table 7, the bases enclosed in each box are the same as the corresponding site of the stem-loop motif sequence of SEQ ID NO: 18 and each base in a white letter enclosed in the black box is a site deleted or substituted compared with the stem-loop motif sequence. In Table 7, each deleted site is indicated by “-”, In the stem-loop motif sequence of SEQ ID NO: 18 of the polynucleotide (d), U at 7^{th} base and 11^{th} base and A at 15^{th} base are preferably maintained, for example.

The His tag aptamer may be, for example, nucleic acid that includes the following polynucleotide (e) or (f):
(e) polynucleotide that includes abase sequence having at least 60% identity in the base sequence of the polynucleotide (a) or (c) and is bindable to the His peptide; and
(f) polynucleotide that includes a base sequence that hybridizes to the base sequence of the polynucleotide (a) or (c) under stringent conditions or the complementary base sequence thereof and is bindable to the His peptide.

Each of the polynucleotides (e) and (f) may be polynucleotide that is composed of or includes the base sequence. The His tag aptamer may be nucleic acid that includes or is composed of the polynucleotide (e) or (f).

In the polynucleotides (e) and (f), “the base sequence of the polynucleotide (a)” corresponds to any of the base sequences of sequence numbers shown in the polynucleotides (a1) to (a3) besides the base sequence of SEQ ID NO: 17 (the same applies hereinafter), for example. In the polynucleotides (e) and (f), “the base sequence of the polynucleotide (c)” corresponds to any of the listed base sequences of the sequence numbers besides the base sequence of SEQ ID NO: 18 (the same applies hereinafter).

In the polynucleotide (e), the identity is, for example, 70% or more, more preferably 80% or more, yet more preferably 90% or more, 91% or more, 92% or more, 93% or more, 94% or more, yet further more preferably 95% or more, 96% or more, 97% or more, 98% or more, particularly preferably 99% or more. The identity can be calculated under default conditions using BLAST or the like, for example.

The aptamer of the polynucleotide (e) preferably has a stem-loop structure that is substantially identical to the stem-loop structure formed by the base sequence of SEQ ID NO: 18, for example.

In the nucleic acid (f), “hybridizes under stringent conditions” refer, for example, to experimental conditions of hybridization well known to those skilled in the technical field. Specifically, “stringent conditions” refer to conditions in which identification can be performed by hybridizing at 60° C. to 68° C. in the presence of 0.7 to 1 mol/L NaCl and then washing at 65° C. to 68° C. using 0.1 to 2 times as much SSC solution, for example. 1×SSC is composed of 150 mmol/L NaCl and 15 mmol/L sodium citrate.

The aptamer of the polynucleotide (e) preferably has a stem-loop structure that is substantially identical to the stem.-loop structure formed by the base sequence of SEQ ID NO; 18, for example.

Each of the polynucleotides (b) (e) may be a partial sequence of the polynucleotide (a) or (c) and is a sequence of preferably continuous 5-mer to 40-mer, more preferably continuous 8-mer to 30-mer, particularly preferably continuous 10-mer to 12-mer.

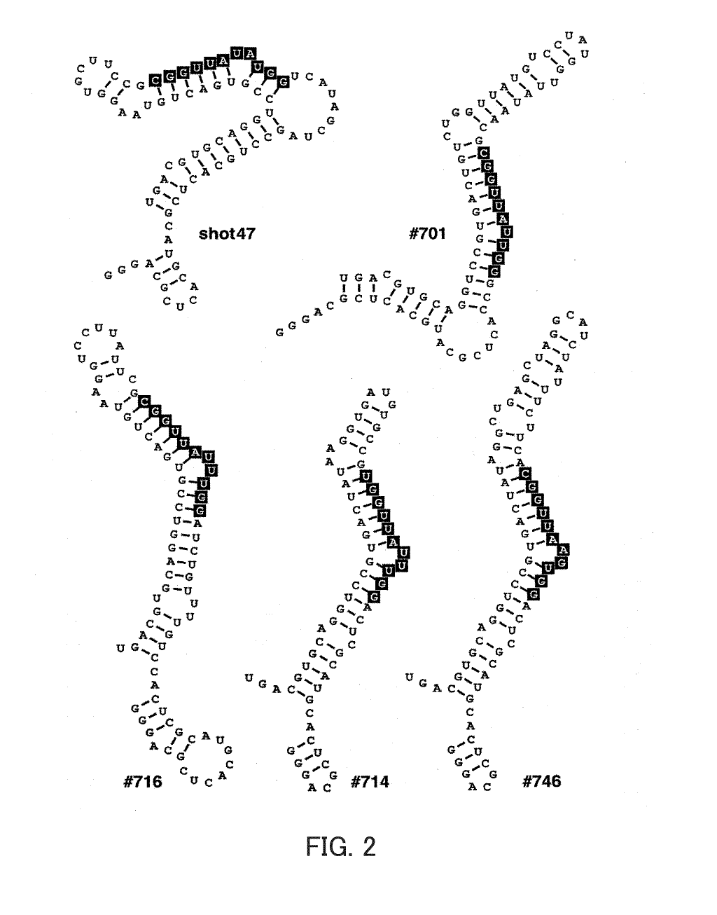

As examples of the His tag aptamer, FIG. 2 shows schematic views of assumable secondary structures of the aptamer shot 47 (SEQ ID NO: 2), the aptamer #701 (SEQ ID NO: 1), the aptamer #716 (SEQ ID NO: 3), the aptamer #714 (SEQ ID NO: 10), and the aptamer #746 (SEQ ID NO: 9). In FIG. 2, each sequence in white letters enclosed in the black box refers to the binding motif sequence represented by SEQ ID NO: 17, which is the consensus sequence among them. In FIG. 2, the binding motif sequence is positioned at the site in which the stem is bent. However, the present invention is not limited thereto.

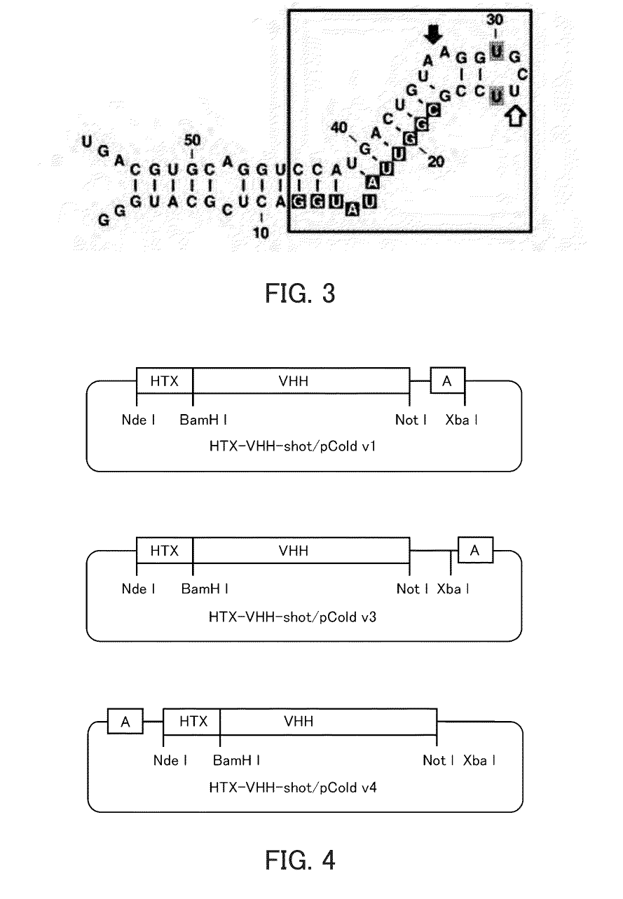

As examples of the aptamer, FIG. 3 shows a schematic view of an assumable secondary structure of the RNA aptamer shot 47 (SEQ ID NO: 2), the RNA aptamer #701 (SEQ NO: 1), the RNA aptamer #714 (SEQ NO: 10), and the RNA aptamer #746 (SEQ ID NO: 9). In FIG. 3, the sequence in white letters enclosed in the black box refers to the consensus sequence represented by SEQ ID NO: 17, and the consensus sequence is positioned at the site in which the stem is bent. However, the present invention is not limited thereto.

Besides the above-mentioned examples, for example, the aptamer can be prepared by the SELEX method or the like, for example.

### Screening Method

The screening method according to the present invention is, as mentioned above, a method for screening for an antibody that is bindable to an antigen or an encoding nucleic acid of the antibody, using the first nucleic acid construct according to the present invention, the method including the steps (A) to (C): (A) a step of expressing the nucleic acid construct to form a complex of a fusion transcript obtained by transcribing the encoding nucleic acid of an antibody candidate (x), the encoding nucleic acid of a peptide tag (y), and the encoding nucleic acid of a nucleic acid molecule (aptamer) (z) and a fusion translation product obtained by translating the encoding nucleic acid of an antibody candidate (x) and the encoding nucleic acid of a peptide tag (y); (B) a step of brining the complex and an antigen into contact with each other; and (C) a step of recovering the complex binding to the antigen.

In the step (A), a first nucleic acid construct according to the present invention, into which the any encoding nucleic acid has been inserted is expressed. Thus, a fusion transcript is obtained by transcribing the antibody candidate-encoding nucleic acid, the tag-encoding nucleic acid, and the aptamer-encoding nucleic acid, and a fusion translation product including the antibody candidate and the tag is obtained by translation. Then, since the aptamer is bindable to the tag, the aptamer in the fusion transcript is bound to the tag in the fusion translation product. Thus, a complex of the fusion transcript and the fusion translation product is formed. The present invention can stably form a complex with a really small molecular size compared with the phage display method, for example. Therefore, for example, it is considered that, in the subsequent step (B), the probability of a contact between the complex and the antigen can be further increased, and nonspecific absorption can be further suppressed.

The complex is formed in vitro. In this case, for example it is preferred that a cell such as a living cell is used, the nucleic acid construct is introduced into the cell, and the nucleic acid construct is expressed in the cell to form the complex. When the nucleic acid construct is introduced into the cell, the number of clones of the nucleic acid construct is increased in the cell, and the complex is formed from each clone, for example. Moreover, the nucleic acid construct may be expressed using a cell-free protein synthesis system or the like, for example. In the present invention, using a cell is preferable because of the simple operation, for example.

The kind of the cell is not particularly limited, and examples thereof include various hosts. Examples of the host include bacteria such as Escherichia such as Escherichia Bacillus such as Bacillus subtilis, Pseudomonas such as Pseadomorias putida, and Rilizobium such as Rhizobium meliloti; and yeast such as Saccharomyces cerevisiae and Schizosaccharomyces pombe. The host can be, for example, preferably Origami (registered trademark) (Merck Ltd.) which is trxB/gor mutant. Further, as the host, for example, animal cells such as a COS cell and a CHO cell; insect cells such as Sf9 and Sf21.; and the like can be used.

The host can be determined suitably according to the kind of the vector of the nucleic acid construct, for example, The combination of the host and the vector is not particularly limited and is, for example, preferably the combination that is superior in induction of expression of peptide (including the meaning of protein), efficiency of transfection, and the like. Specifically, for example, the host is preferably Escherichia coli and the vector is preferably a vector derived from Escherichia coli and is more preferably pCold, which is a cold shock expression vector, because pCold achieves the induction at low temperature. Since the cold expression vector can prevent insolubilization and promote solubilization of peptide expressed in a cell such as Escherichia coli or the like as mentioned above, for example, the expressed peptide can be recovered easily. Further, since the insolubilization of peptide is caused by formation of a peptide inclusion body, for example, it is required to break the inclusion body to take peptide out. However, since such treatment is unnecessary if the above-mentioned vector is used, for example, dissociation of the binding between the fusion transcript and the fusion translation in the complex due to breaking of the inclusion body can be prevented sufficiently. Further, since the cold shock expression vector can suppress the expression of a host-derived peptide by an expression induction at low temperature, for example, peptide derived from the nucleic acid construct can be synthesized efficiently.

Expression of the nucleic acid construct in vitro can be achieved, for example, by transfecting the nucleic acid construct into the cell and performing an induction of expression of peptide with respect to the cell after transfection.

The transfection method of the nucleic acid construct is not particularly limited, and, for example, the method can be set suitably according to the kind of the cell, kind of the vector, and the like. Examples of the transfection method include a protoplast method, a lithium acetate method, the Hanahan method, an electroporation method, a transfection method by infection using a virus vector or the like, a calcium phosphate method, a lipofection method, a transfection method by infection using bacteriophage or the like, a nucleic acid transfection method by ultrasonic, a transfection method using a gene gun, and the DEA.E-dextran method.

The method of inducing peptide expression is not particularly limited, and, for example, the induction of peptide expression can be performed by culturing the cell after transfection. There are no particular limitations on the conditions for the culture, and the conditions can be determined suitably according to the kind of the cell, the kind of the vector, and the like. Specifically, in the case where the cell is Escherichia coli, the culture conditions are, for example, as follows. That is, preferably, the culture temperature is from 20° C. to 40° C. and the culture time is from 0.5 to 6 hours; and more preferably, the culture temperature is from 30° C. to 37° C. and the culture time is from 1 to 3 hours. Examples of the culture medium to be used include an LB culture medium, an NZYM culture medium, a Terrific Broth culture medium, an SOB culture medium, an SOC culture medium, and a 2×YT culture medium.

Further, in the case where the basic vector in the nucleic acid construct is pCold, for example, expression induction at low temperature is possible. Therefore, the culture conditions at the time of expression induction are, for example, as follows. That is, preferably, the culture temperature is from 4° C. to 18° C. and the culture time is from 1 to 24 hours; and more preferably, the culture temperature is from 10° C. to !6° C. and the culture time is from 12 to 24 hours. Further, at the time of culturing, an inducer for inducing expression may be added to a culture medium suitably according to the kind of the cell and the kind of the vector. The inducer is not particularly limited and can be, for example, isopropyl-1-thio-β-galactoside (IPTG). The concentration of the inducer in the culture medium is, for example, from 0.1 to 2 initial and preferably from 0.5 to 1 mmol/L.

Plural nucleic acid constructs each having a different sequence of the any encoding nucleic acid may be introduced into the host, for example. Specifically, for example, the library including plural nucleic acid constructs each having a different random any encoding nucleic acid may be introduced into the cell. By introducing a library of the nucleic acid constructs as described above, for example, the number of clones of each nucleic acid construct is increased in the host, and plural different complexes derived from the respective nucleic acid constructs are formed. Therefore, it is possible to perform screening of plural nucleic acid constructs. Thus, efficiency of screening for the antibody candidate binding to an antigen is further improved.

The step (B) is a step of bringing the complex obtained in the step (A) into contact with an intended antigen. The complex is a complex of a fusion transcript and a fusion translation product as described above. If any peptide in the fusion translation product is bindable to the intended antigen, the complex binds to the intended antigen via the any peptide, for example.

In the step (A), in the case where the complex is formed in the cell, for example, the complex is recovered from the inside of the cell and brought into contact with the antigen. There is no limitation at all on the method of recovering the complex from the cell, and the method can be selected suitably according to the kind of the cell.

Further, in the step (A), in the case of using the cell-free protein synthesis system or the like, for example, the complex is recovered from the cell-free protein synthesis system or the like and brought into contact with the antigen.

There is no limitation at all on the kind of the antigen, and the antigen may be any of peptide such as protein; hormones; nucleic acids; low molecular compounds; organic compounds; inorganic compounds; saccharides; lipids; viruses; bacteria; cells; biological tissues; and the like. For example, the antigen is preferably an immobilized antigen that is immobilized on a solid phase because it can be handled easily, for example. The solid phase is not particularly limited, and examples thereof include plates such as a well plate and a microplate; a chip; a bead such as microsphere; a gel; a resin; a membrane such as a cellulose membrane; a film; a test tube; a micro tube; a plastic container; a cell, a tissue or a fixed paraffin section including the antigen; and a particle.

For example, the solid phase is preferably insoluble. The insoluble material is not particularly limited, and examples thereof include organic resin materials and inorganic materials. The organic resin material may be, for example, a natural material or a synthesized material. Specific examples of the organic resin material include agarose, crosslinking agarose, crosslinking dextran, polyacrylamide, crosslinking polyacrylamide, cellulose, microcrystalline cellulose, crosslinking agarose, polystyrene, polyester, polyethylene, polypropylene, an ABS resin, polyvinyl fluoride, a polyamine-methyl vinyl-ether-maleic acid copolymer, 6-nylon, 6,6-nylon, and latex. Examples of the inorganic material include a glass, a silica gel, diatomaceous earth, titanium dioxide, barium sulfate, zinc oxide, lead oxide, and silica sand. The solid phase may include one or more of the above-mentioned insoluble materials.

In the case where the solid phase is a particle, for example, the particle is preferably a magnetic particle. If the solid phase is a magnetic particle, for example, the magnetic particle can be recovered easily by a magnetic force.

The antigen may be bound to the solid phase directly or indirectly, for example. The immobilization of the antigen on the solid phase may be physical bonding or chemical bonding, for example, and specific examples thereof include adsorption and chemical bonding such as covalent bonding.

There are no particular limitations on the conditions for the contact between the complex and the antigen, and the conditions can be determined suitably according to the kind of the antigen, for example. The conditions are, for example, as follows. Preferably, the temperature is from 4° C. to 37° C., pH is from 4 to 10, and the time is from 10 to 60 minutes; and more preferably, the temperature is from 4° C. to 20° C., pH is from 6 to 9, and the time is from 15 to 30 minutes. The complex and the antigen are brought into contact with each other preferably in a solvent, for example. The solvent is, for example, an aqueous solvent, and as a specific example, any of buffer solutions such as a HEPES buffer solution, a carbonate buffer solution, and a phosphate buffer solution can be used.

The step (C) is a step of recovering the complex that is bound to the antigen. The complex is bound to the antigen via any peptide in the fusion translation product thereof. Therefore, by recovering the complex that is bound to the antigen, the peptide that is bindable to the antigen and the encoding nucleic acid of the peptide can be selected.

The complex that is bound to the antigen may be recovered in the state of binding to the antigen or the state where it is liberated from the antigen, for example.

The recovery of the complex that is bound to the antigen can be performed by washing the antigen, for example. In this manner, for example, the complex that is bound to the antigen can be exclusively recovered by removing the complex that is not bound to the antigen by washing the antigen. In this case, the antigen is preferably immobilized on the solid phase as described above. By washing the solid phase, for example, the complex that is not bound to the antigen that is immobilized on the solid phase is removed. Since the complex that is bound to the immobilized antigen remains on the solid phase, the complex can be recovered in the state of being bound to the antigen. The solid phase is not particularly limited and can be, for example, a base material, and specific examples thereof include base plates such as a plate, a sheet, and a film; containers such as a well plate and a tube; and a bead, a particle, a filter, and gel.

The step (C) may further include a step of liberating the complex that is bound to the antigen from the antigen, for example. The method of liberating the complex from the antigen is not particularly limited.

Furthermore, the step (C) may further include a step of liberating the fusion transcript composing the complex from the complex, for example. The fusion transcript may be liberated after recovering the complex from the antigen or may be liberated from the complex that is bound to the antigen, for example. There is no limitation at all on the method of liberating the fusion transcript from the complex. For example, an eluate containing phenol or the like can be used. As the eluate containing phenol, Trizol (product name, produced by Invitrogen) can be used, for example.

The screening method according to the present invention further includes the following step (D): (D) a step of synthesizing an encoding nucleic acid of any peptide in the antibody candidate, using the fusion transcript in the complex as a template.

In this manner, by further synthesizing the any encoding nucleic acid using the fusion transcript in the complex that is bound to the antigen as a template, specifically the transcript of the any encoding nucleic acid in the fusion transcript as a template, any peptide that is bindable to the antigen and an encoding nucleic acid of the peptide can be identified. The synthesized any encoding nucleic acid may be identified after cloning, for example. With respect to the any encoding nucleic acid, for example, by identifying the base sequence, the amino acid sequence of the any peptide can be identified indirectly.

In the step (D), preferably, the synthesis of the any encoding nucleic acid is performed by the RT (Reverse Transcription)-PCR, for example. Specifically, preferably, the any encoding nucleic acid (DNA) is synthesized by a reverse transcription reaction using the fusion transcript (RNA) as a template, and further, the synthesized any encoding nucleic acid is amplified, for example.

The synthesis of the any encoding nucleic acid may be performed, for example, in the state where the transcript of the any encoding nucleic acid is included in the complex or the state where the fusion transcript is liberated from the complex.

According to the screening method of the present invention, as mentioned above, for example, by the steps (A), (B), and (C), any peptide that is bindable to the antigen and an encoding nucleic acid of the peptide can be selected. Further, by the step (D), the any peptide and the encoding nucleic acid of the peptide can be identified.

Moreover, according to the screening method of the present invention, information on sequences of the any peptide that is bindable to the antigen and the encoding nucleic acid of the peptide can be identified. Therefore, for example, a chimeric antibody, a humanized antibody, a human antibody, and the like can be constructed based on the information.

For example, in the screening method according to the present invention, it is preferred that a first nucleic acid construct according to the present invention to which the any peptide-encodina nucleic acid has been inserted is newly prepared using the any encoding nucleic acid obtained in the step (ID), and the steps (A), (13), and (C) are again performed. It is more preferred that the steps (A), (B), (C), and (D) are repeatedly performed. The number of cycles of repeatedly performing the steps is not particularly limited and is preferably two or more.

As mentioned above, by introducing the library of the nucleic acid constructs into the cells, plural transformants (clones) into which different nucleic acid constructs have been introduced are obtained. Further, by performing the culture in the state where the plural transformants are present, a complex mix in which complexes derived from the respective transformants are present can be obtained. By subjecting this complex mix to the steps (B) and (C), for example, plural complexes each of which is bound to the antigen are recovered. Hence, in the step (D), with respect to the plural complexes recovered in the step (C), for example, the respective any encoding nucleic acids are synthesized. Then, preferably, using the synthesized any encoding nucleic acids, a library of plural nucleic acid constructs into which the respective any encoding nucleic acids have been inserted is produced, and the steps (A), (B), and (C) are performed in the same manner as described above. Thus, any peptide that is bound to the antigen and an encoding nucleic acid of the peptide can be further concentrated, and ally peptide having a good binding affinity to the antigen and an encoding nucleic acid of the peptide can be selected. When the steps (A), (B), (C), and (D) are regarded as 1 cycle, the number of cycles is not particularly limited and is preferably at least 2, for example.

Further, in the case where the library of the nucleic acid constructs is introduced into the cell, for example, the steps (A), (B), and (C) and further the step (D) can be performed after separating plural transformants into each clone or separating plural transformants into plural groups each including several clones. The separation of the transformants into each clone or groups may be performed at the stage in the first cycle or at the stage after the first cycle, for example.

The separated clones as there are can be used as reagent that is bindable to the antigen, or the big number of complexes can be synthesized using the clones, and the complexes can be used after being purified using the tag.

The method of evaluating the binding affinity of the complex to the antigen is not particularly limited. A specific example thereof includes a method in which a labeled anti-tag antibody that is labeled with a labeling substance is used. In this method, for example, after bringing the labeled tag antibody into contact with the substance in which the antigen and the complex are bound to each other, detection of the labeled anti-tag antibody is performed. By detecting the labeled anti-tag antibody, the presence or absence of the tag can be determined. That is, when the complex is bound to the antigen, the labeled anti-tag antibody binds to the tag in the complex. Therefore, by detecting the labeled anti-tag antibody, it can be indirectly determined that the complex is bound to the antigen. On the other hand, when the complex is not bound to the antigen, the tag is not present. Therefore, the labeled anti-tag antibody cannot be detected, and it can be determined that the complex is not bound to the antigen. The label of the labeled anti-tag antibody can be, for example, horseradish peroxidase (HRP), and the detection reagent for detecting the HRP can be, for example, a coloring reagent such as 3,3′,5,5′-tetramethylbenzidine (FMB). Further, for example, depending on whether or not the molecular weight of the target is increased, the binding of the complex can be determined.

In the screening method of the present invention, the evaluation of the binding affinity may be performed in the step (C) or the evaluation of the binding affinity may be performed after selecting the antibody candidate and the antibody candidate-encoding nucleic acid, for example.

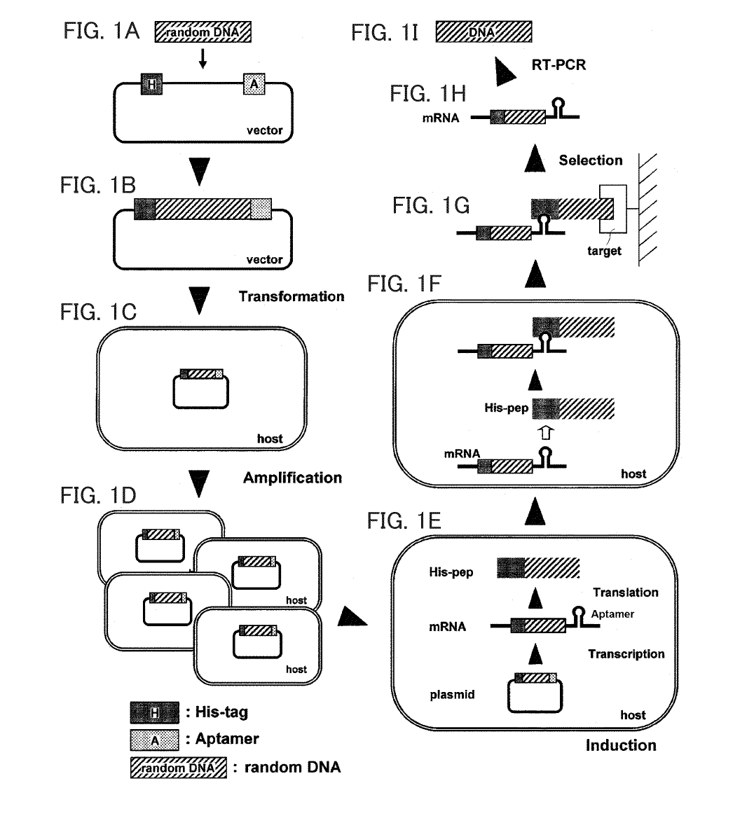

An example of the screening method according to the present invention is described below with reference to FIGS. 1A to 1I FIGS. 1A to 1I are schematic views showing an outline of the screening method in which a complex is formed in vitro. In this example, the first nucleic acid construct according to the present invention is referred to as a vector, and the tag is referred to as the His tag.

First, as shown in FIG 1A, a variable region-encoding nucleic acid (hereinafter, referred to as random DNA) into which any encoding nucleic acid has been inserted is inserted into a vector including a His tag-encoding nucleic acid (H) and an aptamer-encoding nucleic acid (A) to produce a recombinant vector (FIG. 1B). At this time, the His tag-encoding nucleic acid (H) and the random DNA are arranged so that a correct reading frame is obtained.

Then, the recombinant vector is introduced into a host to transform (FIG. 1C). Subsequently, the obtained transformant is amplified (FIG. 1D), and peptide expression is induced (FIG. 1E). As shown in FIG. 1E, by the induction of the expression, first, from the His tag-encoding nucleic acid (H), the random DNA, and the aptamer-encoding nucleic acid (A) in the recombinant vector, a fusion transcript (fusion mRNA) that includes the respective transcripts is formed, and further, on the basis of the fusion transcript, a fusion translation product (fusion peptide: His-pep) that includes the His tag and the random peptide encoded with the random DNA is formed. Then, since an RNA aptamer in the fusion mRNA is bindable to the His tag, the RNA aptamer in the fusion mRNA binds to the fusion peptide to form a complex as shown in FIG. 1F.

Subsequently, the complex and other proteins are taken out from the inside of the transformant and are brought into contact with an antigen immobilized on a solid phase. If the random peptide in the complex is bindable to the antigen, the complex binds to the immobilized antigen via the random peptide (FIG. 1G). In this state, the His tag is bound to the random peptide that is bound to the immobilized antigen, and the fusion mRNA is bound to the His tag via the aptamer. The transcript of the random DNA in this fusion mRNA is mRNA that encodes the random peptide that is bound to the immobilized antigen. Accordingly, by selecting the fusion transcript (FIG. 1H) in the complex that is bound to the immobilized antigen, information on the random peptide that is bound to the immobilized antigen and an encoding nucleic acid of the random peptide can be obtained.

Specifically, RT-PCR is performed using the fusion transcript in the complex as a template, and cDNA is synthesized based on MRNA of the random DNA (FIG. 11). Thus, the information on the base sequence of the encoding nucleic acid of the peptide that is bindable to the antigen and the amino acid sequence of the peptide can be obtained. Further, by introducing the cDNA obtained by the RT-PCR into the vector again (FIG. 1A) and repeatedly performing a series of procedures, the encoding nucleic acid of the peptide that is bindable to the antigen can be further selected.

Next, the screening method according to the present invention is described with reference to the case in which the screening is performed by introducing a plasmid library including random DNA as the nucleic acid construct into Escherichia coli as the cell as an example. Note here that the method described below is merely an example and the present invention is not limited thereby.

First, a plasmid library is introduced into Escherichia coli by electroporation or the like, and the resultant Escherichia coli subjected to shaking culture. A culture medium for the culture can be, for example, an LB medium including ampicillin. For example, the culture of the Escherichia coli is preferably performed until the absorbance at the OD 600 nm becomes 0.5 to 0.6. Further, in the case where the vector of the plasmid library is the cold shock expression vector, for example, a cold shock expression is preferably induced at 15° C. for 18 hours in the presence of 0.5 to 1 mmol/L IPTG.

Next, the cultured Escherichia coli is harvested by centrifugation, the harvested Escherichia coli is suspended in 50 mL of physiological saline including 10 mmol/L EDTA, and the Escherichia coli is again harvested by centrifugation. The recovered Escherichia coli is suspended in 5 mL of a 20 mmol/L HEPES buffer solution containing 20% sucrose and 1 mmol/L EDTA, 10 mg of lysozyme is added to the suspension thus obtained, and the resultant mixture is incubated on ice for 1 hour to dissolve a cell wall. Subsequently, Mg²⁺ is added thereto so as to have a final concentration of 2 mmol/L, and Escherichia coli is harvested by centrifugation. Then, the recovered Escherichia coli is suspended in 50 mL of physiological saline containing 0.1 mmol/L magnesium acetate, and centrifugation is again performed to recover spheroplast.

The recovered spheroplast is promptly suspended in 2.5 to 5 mL of 20 mmol/L HEPES buffer solution containing 0.05% to 0.5% Triton (registered trademark)-X100, 0.1 mmol/L magnesium acetate, 0.1 mg/mL tRNA, 0.1% HSA or BSA (RNase free), and a protease inhibitor to be subjected to bacteriolysis. Thereafter, genomic DNA of the Escherichia coli is sheared by mechanical shearing or DNase 1. Then, NaCl is added thereto so as to have a final concentration of 150 mmol/L, the resultant mixture is allowed to stand for 5 minutes and is then centrifuged to obtain a supernatant including the complex. The supernatant can be stored at −80° C. until binding to the target is evaluated, for example.

The supernatant including the complex is brought into contact with an antigen and then incubated at 4° C. for 10 to 30 minutes, The antigen is preferably immobilized on a solid phase as mentioned above. The solid phase on which the antigen is immobilized is not particularly limited, and examples thereof include a gel on which the antigen is immobilized, a plastic container on which the antigen is immobilized, a cell including the antigen, and a tissue including the antigen. It is preferred that the solid phase on which the antigen is immobilized is preliminarily blocked with HAS or BSA which is the same as those added at the time of bacteriolysis, for example.

Subsequently, the solid phase is washed with a washing liquid to remove the complex. that is not bound to the antigen. The washing liquid can be, for example, a 20 mmol/L HEPES buffer solution containing 0.05% to 0.5% Triton (registered trademark)-X100, 0.1 mmol/L magnesium acetate, and 100 to 150 mmol/L NaCl.

Next, the complex is liberated and recovered from the antigen immobilized on the solid phase by the eluate. Examples of the eluate include a buffer solution containing a denaturant such as Trizol (invitrogen), isogen (Wako), 8 mol/L urea, 6 mol/L guanidine, or 1% SDS and a. buffer solution further containing 0.05 to 0.5% Triton (registered trademark)-X100 and 1 to 10 mmol/L EDTA in addition to the denaturant. The buffer solution is not particularly limited and can be, for example, a Tris buffer solution.

Then, a fusion transcript (RNA) is purified from the obtained complex. For the purification of RNA, Trizol (product name, produced by invitrogen) or the like can be used, for example. Further, in the case of ethanol precipitation, a precipitation aid such as tRNA, glycogen, or Ethatinmate (product name, produced by Nippongene) is preferably used, for example. It is preferred that, in the purification of RNA, incubation at 37° C. for 30 minutes was performed using RNase free DNase, and thereafter, phenol-chloroform extraction and ethanol precipitation are performed, for example.

Subsequently, using the purified RNA as a template, cDNA is synthesized by RT-PCR. It is preferred that the synthesized cDNA is subjected to PCR to form a complementary double-stranded cDNA, for example. In the case where plural random DNAs are used as any encoding nucleic acids, for example, there is a possibility that plural cDNAs each having the 5′ side region and the 3′ side region in the any encoding nucleic acid that are in common among the cDNAs and having a sequence of an intermediate region that differs among the cDNAs are synthesized by the RT-PCR and heteroduplex cDNA is formed. Hence, it is preferred that the cDNA synthesized by RT-PCR is further subjected to PCR to elongate a complementary strand and to amplify complementary double-stranded cDNA. The method of amplifying the complementary double-stranded cDNA is not particularly limited and can be performed by further adding a forward primer and a reverse primer to the reaction solution of the RT-PCR, performing thermal denaturation, and repeatedly performing the annealing reaction and the elongation reaction after denaturation. The amount of the primer added to the reaction solution is not particularly limited. It is preferred that each primer is added to the reaction solution so as to have a final concentration of 10 mmol/L, for example. Further, preferably, the thermal denaturation is performed, for example, by treating at 95° C. for 30 seconds and then treating at 94° C. for 3 minutes; the annealing reaction is performed, for example, at 63.2° C. for 3 minutes; the elongation reaction is performed, for example, at 72° C. for 3 minutes. It is preferred that the annealing reaction and the elongation reaction are repeated 5 times, for example. In this manner, the complementary double-stranded cDNA can be obtained.

It is preferred that the obtained double-stranded cDNA is again inserted into a vector such as the above-mentioned plasmid and a series of procedures described above is performed repeatedly. Thus, any plasmid that is bindable to an antigen can be further selected.

Further, for improving selection efficiency, for example, after introducing the nucleic acid construct into Escherichia coli, the Escherichia coli may be dispensed to a multi-well plate to limit clones. Specifically, the Escherichia coli is amplified in the plate, some of the cultured Escherichia coli are stored, and then an expression induction and bacteriolysis are performed. The bacteriolysis can be performed, for example, by adding a 20 mmol/L HEPES buffer solution containing 0.05% to 0.5% Triton (registered trademark)-X100, 1 mmol/L EDTA, 2 mg/mL lysozyme, and 1 mg/mL DNase after harvesting the Escherichia coli in the plate. The bacterial lysate thus prepared is added to a plate on which the antigen is immobilized to cause the complex in the bacterial lysate to bind to the antigen. Thereafter, the plate is washed with a 20 mmol/L HEPES buffer solution containing 0.05% to 0.5% Triton (registered trademark)-X100 and 1 mmol/L EDTA, and then the complex that is bound to the antigen is detected using an HRP-labeled anti-His tag antibody or the like as mentioned above. Thus, the well including a plenty of clones that form complexes each binding to the antigen is specified. Further, from the preliminarily stored Escherichia coli, the Escherichia coli of the corresponding well is selected and amplified, and selection is subsequently performed by a series of procedures.

### Second Nucleic Acid Construct

A second nucleic acid construct according to the present. invention is a nucleic acid construct into which the any encoding nucleic acid can be inserted, and for example, by inserting the any encoding nucleic acid which is set by an experimenter, the first nucleic acid construct according to the present invention can be prepared. That is, the second nucleic acid construct according to the present invention is a nucleic acid construct for expressing an antibody candidate, the nucleic acid construct including the following encoding nucleic acids (x′), (y), and (z): (x′) an encoding nucleic acid of a variable region of an antibody, into which an encoding nucleic acid of any peptide can be inserted; (y) an encoding nucleic acid of a peptide tag; and (z) an encoding nucleic acid of an aptamer that is bindable to the peptide tag, wherein the encoding nucleic acids (x′), (y), and (z) are linked with one another so that the encoding nucleic acids (x′), (y), and (z) are transcribed as a fusion transcript, and the encoding nucleic acids (x′) and (y) are translated as a fusion translation product.

The configuration of the second nucleic acid construct according to the present invention is not at all limited as long as the any encoding nucleic acid can be inserted into the encoding nucleic acid (x′). The second nucleic acid construct according to the present invention is the same as the first nucleic acid construct unless otherwise shown and can be described with reference to the description of the first nucleic acid construct.

In the second nucleic acid construct according to the present invention, the variable region and the variable region-encoding nucleic acid can be described with reference to the description of the first nucleic acid construct.

The encoding nucleic acid (x′) may be the variable region-encoding nucleic acid or the variable region-encoding nucleic acid with a partial deletion. In the former case, the any encoding nucleic acid may be inserted into the end and/or the inside of the variable region-encoding nucleic acid (x′) by addition. For example, it is also possible that at least partial region of the variable region-encoding nucleic acid (x′) is deleted, and the any encoding nucleic acid is inserted into the deleted site (substitution). On the other hand, in the latter case, the any encoding nucleic acid may be inserted into a deleted site of the variable region-encoding nucleic acid (x′) by addition. By inserting the any encoding nucleic acid into the second nucleic acid construct according to the present invention as described above, the first nucleic acid construct can be prepared.

### Screening Kit

The screening kit according to the present invention is a kit used for the screening method according to the present invention and includes the second nucleic acid construct according to the present invention. The screening kit according to the present invention is characterized by including the second nucleic acid construct according to the present invention, and other configurations and the like are not at all limited. The screening kit according to the present invention may include the first nucleic acid construct according to the present invention, for example.

The kit according to the present invention may further include a living cell to be introduced with the nucleic acid construct. Further, each of the kits according to the present invention may include a reagent, an instruction manual, and the like for introducing the nucleic acid construct into the living cell, for example.

### EXAMPLES

Next, the examples of the present invention are described. However, the present invention is not limited by the following examples. Commercially available reagents were used based on the protocols thereof unless otherwise shown.

### Example 1

A fusion protein (HTX-VHH) of a tag peptide and VHH was expressed, and a plasmid vector in which an aptamer to the tag is bound to the fusion protein was constructed.

### (1) VIM Artificial Gene

Based on an amino acid sequence of VHH derived from llama, the following VHH artificial gene (SEQ ID NO: 57) including no CDR3 region was synthesized.

TABLE 8
SEQ ID NO: 57
ATG CGG GGT TCT CAT CAT CAT CAT CAT CAT GGT ATG
GCT AGC ATG ACT GGT GGA CAG CAA ATG GGT CGG GAT
CTG TAC GAC GAT GAC GAT AAG GAT CGA TGG GGA TCC
CAG GTG CAG CTA CAA GAA TCT GGG GGT GGC CTG GTG
CAG GCG GGC GGT TCC CTG CGT CTC TCC GCG GCA GCC
TCT GGC CGC ACC TTC AGT AGC TAT GGC ATG GGC TGG
TTT CGT CAG GCT CCG GGC AAA GAA CGT GAA TTC GTC
GCA GCG ATC AGC TGG TCT GGC GGT TCC ACC TAC TAT
GCA GAC AGC GTG AAA GGC CGC TTC ACC ATC TCC CGG
GAC AAC GCG AAA AAC ACC GTG TAC CTG CAA ATG AAC
AGT CTG AAA CCG GAA GAC ACG GCC GTT TAT TAC GCT
GCA GCG GTT TCC AGC GGC CGC TAA

In the sequence, a double underlined part on the 5′ side represents an encoding nucleic acid of CDR1 (ACCTTCAGTAGCTATGGCATGGGC: SEQ ID NO: 58), and a double underlined part on the 3′ side represents an encoding nucleic acid sequence of CDR2 (TTCGTCGCAGCGATCAGCTGGTCTGGCGGTTCCACCTAC: SEQ ID NO: 59). Among single underlined parts on the 3′ side, an upstream part represents a recognition site of PstI, a downstream part represents a recognition site of NotI, and a random sequence is inserted between these recognition sites as mentioned below. A single underlined sequence from the 5′end represents an encoding DNA of HTX tag derived from pRSET (trade name, Invitrogen). The HTX tag is tag peptide in which a His tag, a T tag, and an Xpress tag are linked with one another. An encoding DNA of the His tag is an encoding DNA (ATGCGGGGTTCTCATCATCATCATCATCATGGT: SEQ ID NO: 61) of the His tag (MRGSHHHHHHG: SEQ ID NO: 60) that includes continuous 6 histigines. An encoding DNA of the T tag is an encoding DNA. (ATGGCTAGCATGACTGGTGGACAGCAAATGGGT: SEQ ID NO: 63) of a peptide tag (MASMTGGGGMG: SEQ ID NO: 62) that includes a T7 gene 10 leader including 10 amino acid residues. An encoding DNA of the Xpress tag is an encoding DNA (CGGGATCTGTACGACGATGACGATAAGGATCGATGGGGATCC: SEQ ID NO: 155) of Xpress ™Epitope (RDLYDDDDKDRWGS: SEQ ID NO: 64) that includes 14 amino acid residues.

### (2) Construction of Plasmid Vector Including VHH Artificial Gene

Three kinds of plasmid vectors obtained by inserting an encoding DNA of an aptamer that is bindable to the His tag into different sites were constructed.

### (2-1) HTX-VHH-shot/pColdv1

DNA represented by SEQ ID NO: 156 was inserted into NdeI-Xbal of pCold (registered trademark) 4 vector (trade name, Takara Bio Inc.). In the following sequence, the double underlined T at the 3′ side was substituted by A at the time of the insertion. In the following sequence, a region indicated by capital letters is the VHH artificial gene, and a single underlined part is an encoding DNA (SEQ ID NO: 158) of an aptamer of SEQ ID NO: 157, and the encoding DNA includes an encoding DNA of shot47sss of SEQ ID NO: 102. In the following sequence, regions each enclosed in the box are restriction enzyme recognition sites, and the upstream region represents PstI and the downstream region represents NotI. The vector obtained as described above is referred to as HTX-VHH-shot/pColdv1.

TABLE 9
SEQ ID NO: 156
catATGCGGGGTTCTCATCATCATCATCATCATGGTATGGCTAGCATGACTGGTGGACAGCAAATGGGTCGGGATC
TGTACGACGATGACGATAAGGATCGATGGGGATCCCAGGTGCAGCTACAAGAATCTGGGGGTGGCCTGGTGCAGGC
GGGCGGTTCCCTGCGTCTCTCCGCGGCAGCCTCTGGCCGCACCTTCAGTAGCTATGGCATGGGCTGGTTTCGTCAG
GCTCCGGGCAAAGAACGTGAATTCGTCGCAGCGATCAGCTGGTCTGGCGGTTCCACCTACTATGCAGACAGCGTGA
AA GGC CGC TTC ACC ATC TCC CGG GAC AAC GCG AAA AAC ACC GTG
TAC CTG CAA ATG AAC AGT CTG AAA CCG GAA GAC ACG GCC GTT TAT

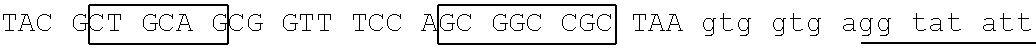

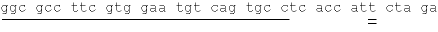

### (2-2) HTX-VHH-shot/pColdv3

DNA represented by SEQ ID NO: 69 was inserted into NdeI-ClaI of pCold (registered trademark) 4 vector (trade name, Takara Bio Inc.). In the following sequence, a region indicated by capital letters is the VHH artificial gene, and a single underlined part is an aptamer-encoding DNA. In the following sequence, regions each enclosed in the box are restriction enzyme recognition sites, and the upstream region represents PstI and the downstream region represents NotI. The vector obtained as described above is referred to as HTX-VHH-shot/pColdv3.

TABLE 10
SEQ ID NO: 69
cat ATG CGG GGT TCT CAT CAT CAT CAT CAT CAT GGT ATG GCT AGC
ATG ACT GGT GGA CAG CAA ATG GGT CGG GAT CTG TAC GAC GAT GAC
GAT AAG GAT CGA TGG GGA TCC CAG GTG CAG CTA CAA GAA TCT GGG
GGT GGC CTG GTG CAG GCG GGC GGT TCC CTG CGT CTC TCC GCG GCA
GCC TCT GGC CGC ACC TTC AGT AGC TAT GGC ATG GGC TGG TTT CGT
CAG GCT CCG GGC AAA GAA CGT GAA TTC GTC GCA GCG ATC AGC TGG
TCT GGC GGT TCC ACC TAC TAT GCA GAC AGC GTG AAA GGC CGC TTC
ACC ATC TCC CGG GAC AAC GCG AAA AAC ACC GTG TAC CTG CAA ATG

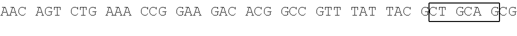

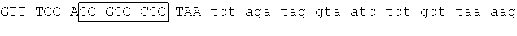

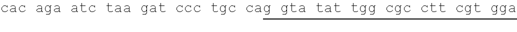

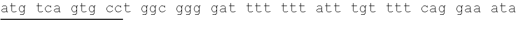

aat aat cga t

### (2-3) HTX-V111I-shot/pColdv4

DNA represented by SEQ ID NO: 70 was inserted into NheI-Xbal of pCold (registered trademark) 4 vector (trade name, Takara Bio Inc.). In the following sequence, a double underlined C at the 6^{th} position from the 5′ end was substituted by T at the time of the insertion.

In the following sequence, a single underlined part represents an aptamer-encoding DNA, and a region indicated by capital letters represents the VHH artificial gene. In the following sequence, regions each enclosed in the box represent restriction enzyme recognition sites, and the upstream region represents PstI and the downstream region represents NotI. The vector obtained as described above is referred to as HTX-VHH-shot/pColdv4.

TABLE 11
SEQ ID NO: 70

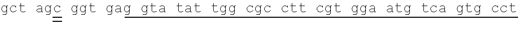

cac cgc tag cgc ata tcc agt gta gta agg caa gtc cct tca aga
gtt atc gtt gat acc cct cgt agt gca cat tcc ttt aac gct tca
aaa tct gta aag cac gcc ata tcg ccg aaa ggc aca ctt aat tat
taa gag gta ata cca tAT GCG GGG TTC TCA TCA TCA TCA TCA TCA
TGG TAT GGC TAG CAT GAC TGG TGG ACA GCA AAT GGG TCG GGA TCT
GTA CGA CGA TGA CGA TAA GGA TCG ATG GGG ATC CCA GGT GCA GCT
ACA AGA ATC TGG GGG TGG CCT GGT GCA GGC GGG CGG TTC CCT GCG
TCT CTC CGC GGC AGC CTC TGG CCG CAC CTT CAG TAG CTA TGG CAT
GGG CTG GTT TCG TCA GGC TCC GGG CAA AGA ACG TGA ATT CGT CGC
AGC GAT CAG CTG GTC TGG CGG TTC CAC CTA CTA TGC AGA CAG CGT
GAA AGG CCG CTT CAC CAT CTC CCG GGA CAA CGC GAA AAA CAC CGT
GTA CCT GCA AAT GAA CAG TCT GAA ACC GGA AGA CAC GGC CGT TTA

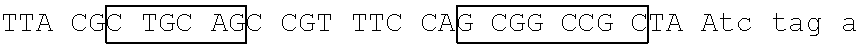

Schematic views of FIG. 4 schematically show the three kinds of plasmid vectors. In the HTX-VHH-shot/pCold v1, the aptamer DNA was inserted downstream of the VHH gene and upstream of a terminator. In the HTX-VHH-shot/pCold v3, the aptamer DNA was inserted downstream of the VHH gene in a terminator region. In the HTX-VHH-shot/pCold v4, the aptamer DNA was inserted upstream of the encoding DNA of the HTX tag.

(3) Production of library vectors

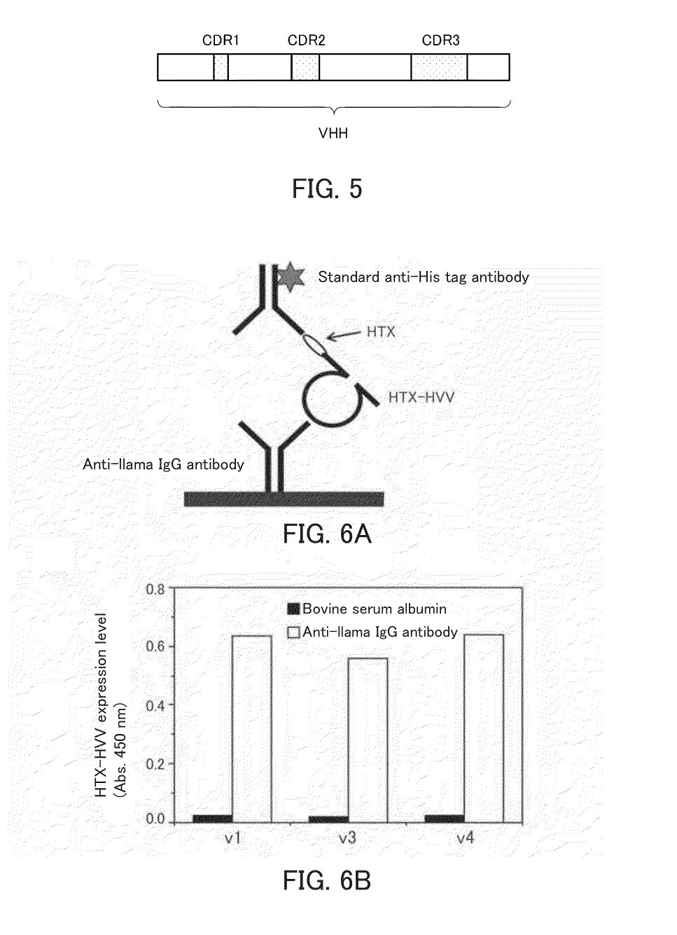

FIG. 5 schematically shows a configuration of VHH. As shown in FIG. 5, the VHH includes a CDR1 region, a CDR2 region, and a CDR.3 region. The inside of an encoding DNA of the CDR.3 region of the of each plasmid vector produced in the item “2,” above was substituted by a random sequence to produce each library vector having the random sequence.

(3-1) Insert for library

First, oligonucleotide A1 (SEQ ID NO: 71) including a random region (underlined part), oligonucleotide A2 (SEQ ID NO: 72) including a random region (underlined part), complementary oligonucleotide El (SEQ ID NO: 73), and complementary oligonucleotide B2 (SEQ ID NO: 74) were synthesized. In the following sequences, V was A, C or G, N was A, C,

G, or T, and K was G or T. A codon was set to VNK, so that appearances of a stop codon, a Cys residue, a Phe residue having high hydrophobicity, and a Trp residue were suppressed, and appearance ratios of the other amino acids were relatively even. In each of the oligonucleotides A1 and A2, a codon of tyrosine was set to a part enclosed in the box.

TABLE 12
Oligonucleotide A1 (SEQ ID NO: 71)

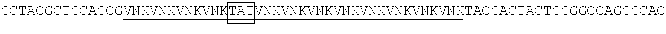

Oligonucleotide A2 (SEQ ID NO: 72)

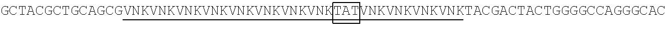

Oligonucleotide B1 (SEQ ID NO: 73)
CACTTAGCGGCCGCTGGAAACGGTCACCTGGGTGCCCTGGCCCCAGTAGTCGTA
Oligonucleotide B2 (SEQ ID NO: 74)
CACTTAGCGGCCGCTCACGTAGGCTTGCTGCAAGTCGATGGTGCAACTCTACCGCTGGAAACGGTAACCTGAGTGC
CCTGGCCCCAGTAGTCGTA

50 pmol of the oligonucleotide A1, 50 pmol of the oligonucleotide A2, and 1000 pmol of the oligonucleotide B1 or B2 were mixed to prepare a total of 100 μL of a reaction solution using TaKaRa Ex Tag (trade name, Takara Bio Inc.). The reaction solution was heated at 98° C. for 30 seconds, and one cycle of treatment at 60° C. for 1 minute and 72° C. for 1 minute was repeated a total of 5 cycles. DNA was recovered from the reaction solution by ethanol precipitation and was treated with 50 units of exonuclease I (Takara Bio Inc.) at 37° C. for 7 hours to digest a single-stranded DNA. The recovered DNA was subjected to phenol-chloroform extraction and thereafter subjected to ethanol precipitation. The resultant double-stranded DNA was digested with 30 units of PstI and 30 units of NotI at 37° C. for 18 hours. A digested fragment was separated by electrophoresis using 3% NuSieve GTG agarose (Takara Bio Inc.), and a band at around 100 bp was cut out and was subjected to DNA extraction using AgarACE enzyme (trade name, Promega KK.). The extracted DNA was further subjected to phenol extraction, phenol-chloroform extraction, and ethanol precipitation. The DNA thus obtained as it was or the DNA thus obtained after being subjected to an enzyme treatment using alkaline phosphatase (calf intestine) (trade name, Takara Bio Inc.), phenol-chloroform extraction, and ethanol precipitation was used as an insert for library.

### (3-2) Vector for Library

20 μg of the plasmid vector (HTX-VHH-shot/pColdv1, HTX-VHH-shot/pColdv3, or HTX-VHH-shot/pColdv4) produced in the item “2.” above was treated with 30 units of PstI at 37° C. for 8 hours. Thereafter, 30 units of NotI were added thereto, which was then digested at 37° C. for 18 hours. The digested fragment was separated by electrophoresis using 3% NuSieve GTG agarose (Takara Bio Inc.), a band at around 5000 bp was cut off, and DNA was extracted using AgarACE enzyme (trade name, Promega KK.). The extracted DNA was further subjected to phenol extraction, phenol-chloroform extraction, and ethanol precipitation, and the obtained DNA fragment was used as a vector for library. Hereinafter, a vector into which the oligonucleotide including a random region is not inserted is referred to as a “vector for library, and a vector into which the oligonucleotide including a random region is inserted is referred to as a “library vector”.

(3-3) Insertion of Insert for Library into vector for Library

0.2 μg of the insert for library and 1 μg of the vector for library were mixed, and the resultant mixture was subjected to a ligation reaction at 14° C. for 18 hours using 1750 units of T4 DNA ligase (Takara Bio Inc.). The reaction was performed in a 6 mmol/L tris buffer solution (pH7.5) containing 0.1 mg/mL Bovine serum albumin, 7 mmol/L 2-mercaptoethanol, 0.1 mmol/L ATP, 2 mmol/L dithiothreitol, 1 mmol/L spermidine, 5 mmol/L NaCl, and 6 mmol/L MgCl₂. By this reaction, the insert for library was inserted into the vector for library, and thus, a library vector into which a random region was inserted was constructed.

10 μg of tRNA (derived from Saccharomyces cerevisiae, Sigma-Aldrich) was added to the reaction solution, and the resultant mixture was subjected to phenol-chloroform extraction and ethanol precipitation and then dissolved in 10 μL of TE. The whole amount of the obtained solution was mixed in Escherichia coli to transform. The transformation was performed using 100 μL Ecoli DH5α Electoro-cells (Takara Bio Inc.) as Escherichia coli and an electroporator (BRL Life Technologies, Inc.) under the conditions at 380 V and 4 kΩ/330 μF. The transformed Escherichia coli was suspended in 6 mL of SOC, the suspention thus obtained was subjected to shaking culture at 37° C. for 1 hour, and thereafter, a small amount of the culture solution thus obtained was collected to measure complexity of the library. 14 mL of LB containing ampicillin with a final concentration of 100 μg/mL was added to the remaining culture solution, which was then subjected to shaking culture at 37° C. for 5 hours. The obtained culture solution was divided into 4 mL each, and 0.28 mL of DMSO was added to each 4 mL of the culture solution, and immediately after the addition, the resultant solution was frozen in liquid nitrogen and stored at −80° C. The complexity of the library per performing this method one time was 5×10⁶ to 10×10⁶ cfu.

### (4) Selection of Library Vector

### (4-1) Expression of Protein

A library of frozen Escherichia coli into which various library vectors were transformed, produced in the item “3.” above was promptly dissolved in 46 mL of LB containing ampicillin with a final concentration of 100 μg/mL, The resultant solution was then subjected to shaking culture at 37° C. until the absorbance at 600 nm became 0.5. The culture solution thus obtained was cooled at 10° C. for 30 minutes, IPTG with a final concentration of 0.5 mmol/L was then added to the culture solution, and thereafter, the culture solution was subjected to shaking culture at 10° C. for 18 hours. The culture solution thus obtained was centrifuged at 6,500×g and 4° C. for 10 minutes to collect bacterial cells. The collected bacterial cells were suspended in 50 mL of a saline solution containing 10 mmol/L EDTA, and the suspension thus obtained was centrifuged at 6,500×g a and 4° C. for 10 minutes to wash the bacterial cells. The bacterial cells were suspended in 5 mL of a 20 mmol/L HEPES buffer solution (pH7.6) containing 20% sucrose and 1 mmol/L EDTA, and 100 μL of 0.1 g/mL egg-white lysozyme (Sigma-Aldrich) was added to the suspension, and the resultant suspension was stirred and treated for 1 hour on ice. Furthermore, 5 μL of 1 mol/L magnesium acetate containing 1 mol/L MgCl₂ was added to the suspension, which was then centrifuged at 6,500×g and 4° C. for 10 minutes to collect spheroplast. The collected spheroplast was suspended in 50 mL of a 20 mmol/L HEPES buffer solution (pH7.6) containing 0.1 mmol/L magnesium acetate and 0.9% NaCl, and the resultant suspension was allowed to stand still for 5 minutes on ice and thereafter centrifuged to wash. A bacteriolytic reagent was added to a precipitate of the spheroplast after the washing, and the resultant mixture was vigorously stirred at 4° C. Thus, bacteriolysis was performed. As the bacteriolytic reagent, 2 mL of a 20 mmol/L HEPES buffer solution (pH7.6) containing 100 μg/mL tRNA (derived from Saccharomyces cerevisiae, Siama-Aldrich), 0.1% human serum albumin (Sigma-Aldrich), 50 units of RNase A inhibitor (TOYOBO CO., LTD.), 210 units of DNase I (Invitrogen), 1/6 pieces of complete mini EDTA-free proteinase inhibitor cocktail tablets (Roche Ltd.), 0.5% TritonX (registered trademark)-100, and 0.1 mmol/L magnesium acetate was used. The bacterial lysate was aspirated and discharged with a syringe provided with a 27-gauge injection needle, and a disruption of spheroplast was accelerated, and a genomic DNA was sheared. Thereafter, the resultant mixture was allowed to stand still for 5 minutes on ice and centrifuged at 17,000×g for 10 minutes to collect a supernatant.

### (4-2) Measurement of Protein Expression Level

The expression level of each HTX-VHH protein was measured by sandwich ELISA shown below. The schematic view of the sandwich ELISA is shown in FIG. 6A. As shown in FIG. 6A, the expression level of the HTX-HVV protein can be measured by trapping the HTX-HVV protein with an immobilized anti-llama IgG antibody and causing a labeled anti-His tag antibody to bind to a His tag of the HTX-HVV protein.

80 μL a well of a 50 mmol/L carbonate buffer solution (pH9.0) containing 5 μg/mL goat anti-llama IgG antibody was added to a 96-well plate (ASAHI GLASS CO., LTD.), and the plate was allowed to stand still for 3 hours at room temperature to cause the antibody to be absorbed. Thereafter, each well was blocked with 200 μL of a 20 mmol/L HEPES buffer solution (pH7.6) containing 1% human serum albumin (Sigma-Aldrich) and 0.9% NaCl. On the other hand, as a negative control, wells which were subjected to only the blocking were prepared. 200 μL each of the bacterial lysate of the library of Escherichia coli using various library vectors, obtained in the item “4.” above were added to each well, which was then cultured at 4° C. for 1 hour. The well was washed four times with a tris buffer solution (pH7.6) containing 0.1% Tween-20 and 0.9% NaCl, and a 20 mmol/L tris buffer solution (pH7.6) containing horseradish peroxidase-labeled anti-His tag antibody (QIAGEN) diluted 2000-hold, 0.2% Bovine serum albumin, and 0.9% NaCl was added to the well to perform a reaction for 1 hour at room temperature. The well was washed four times with a tris buffer solution (pH7.6) containing 0.1% Tween-20 and 0.9% NaCl, and then, 1 Step Ultra TMB-ELISA(trade name, Thermo Fisher Scientific K.K.) was added to the well to cause a coloring reaction to be performed. The reaction was terminated with sulfuric acid, and thereafter, an absorbance at 450 nm was measured.

The results of these are shown in FIG. 6B. FIG. 6B is a graph showing expression levels of the HTX-VHH proteins. As shown in FIG. 6B, expressions of the HTX-VHH proteins were determined in all of the cases of using the respective library vectors.

(4-3) Measurement of Binding mRNA

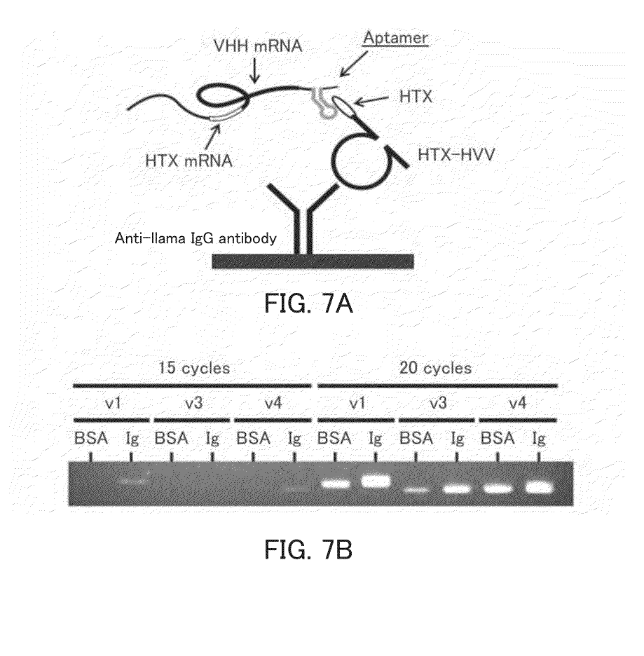

The amount of mRNA binding to each HTX-VHH protein was measured by the following method. A principle of the measurement of mRNA is shown in a schematic view of FIG. 7A. When each library vector is expressed in Escherichia coli, fusion mRNA including an aptamer is transcribed, and the HTX-HVV protein is translated. The aptamer binds to the His tag, so that the fusion mRNA and the HTX-HVV protein are bound to each other via the binding between the aptamer and the His tag. Thus, as shown in FIG. 7A, the fusion mRNA binding to the HTX-HVV protein can be measured by trapping the HTX-HVV protein with an immobilized anti-llama IgG antibody.

120 μL per a well of a 50 mmol/L carbonate buffer solution (pH9.0) containing 5 μg/mL goat anti-llama IgG antibody was added to a 96-well plate (ASAHI GLASS CO., LTD.), and the plate was allowed to stand still for 3 hours at room temperature to cause the antibody to be absorbed. Thereafter, each well was blocked with 200 μL of a 20 mmol/L HEPES buffer solution (pH7.6) containing 1% human serum albumin (Sigma-Aldrich) and 0.9% NaCl. On the other hand, as a negative control, wells which were subjected to only the blocking were prepared. 200 μL each of the bacterial lysate of the library of Escherichia coli using various library vectors, obtained in the item “4,” above were added to each well, which was then cultured at 4° C. for 1 hour. The well was washed four times with a 20 mmol/L HEPES buffer solution (pH7.6) containing 0.5% TritonX-100 and 0.1 mmol/L magnesium acetate, and 150 μL of a Trizol reagent (trade name, Invitrogen) was added to the well to collect mRNA. 1 μL of Ethatinmate (trade name, NIPPON GENE CO., LTD,) as a carrier for alcohol precipitation was added to the collected mRNA, and RNA was purified according to the protocols of the alcohol precipitation. The obtained RNA was treated at 37° C. for 30 minutes using 5 units of DNase (Promega KK.) and then subjected to phenol-chloroform extraction and ethanol precipitation. Thus, purified RNA was obtained.

RT-PCR was performed by a One-step RT-PCR kit (trade name, QIAGEN) using the whole amount of the purified RNA. The conditions include an annealing temperature of 55° C. and the number of cycles of 15 or 20. As primers, the following two kinds of primers were used in combination (SEQ ID NOs: 75 and 76).

Primer C1
(SEQ ID NO: 75)
GGCTAGCATGACTGGTGGACAGCAAA
Primer C2
(SEQ ID NO: 76)
GGCAGGGATCTTAGATTCTG

A reaction solution of the RT-PCR was subjected to electrophoresis using 1.5% agarose, and a PCR fragment was stained with ethidium bromide. The result of this electrophoresis is shown in a photograph of FIG. 7B. In FIG. 7B, v1, v3, and v4 represent the respective kinds of the used library vectors, BSA represents a result of the negative control, Ig represents a result of the immobilized goat anti-llama IgG antibody. As shown in the results obtained in the case of 20 cycles of FIG. 7B, in each plasmid vectors, the result of Ig showed an intense band compared with the result of BSA.

These results show that a complex of a fusion protein and fusion mRNA can be formed even if the aptamer DNA is inserted into any of the sites.

### Example 2

Screening for a variable region binding to human intelectin-1 was performed using the HTX-VHH-shot/pColdv1 produced in Example 1.

(1) Construction of library

As a vector for library, the HTX-VHH-shot/pColdv1 was used. Moreover, an insert for library was prepared from the oligonucleotide A1 (SEQ ID NO: 71), the oligonucleotide A2 (SEQ ID NO: 72), and the complementary oligonucleotide B1 (SEQ ID NO: 73) in the same manner as in Example 1. Then, in the same manner as in the item “3, (3-3)” of Example 1, a library vector including a random region inserted thereinto was constructed, and a library of frozen Escherichia coli obtained by transforming the library vector was prepared.

A library of frozen Escherichia coli was promptly dissolved in 100 mL of LB containing ampicillin with a final concentration of 100 μg/ml. The resultant solution was then subjected to shaking culture at 37° C. until the absorbance at 600 nm became 0.6. The culture solution thus obtained was cooled at 10° C. for 30 minutes, IPTG with a final concentration of 1 mmol/L was then added to the culture solution, and thereafter, the culture solution was subjected to shaking culture at 10° C. for 1 hour. The culture solution thus obtained was centrifuged at 6,500×g and 4° C. for 10 minutes to collect bacterial cells. The collected bacterial cells were suspended in 50 mL of a saline solution containing 10 mmol/L EDTA, and the suspension thus obtained was centrifuged at 6,500Δg and 4° C. for 10 minutes to wash the bacterial cells. The bacterial cells were suspended in 5 mL of a 20 mmol/L HEPES buffer solution (pH7.6) containing 20% sucrose and 1 mmol/L EDTA, and 100 μL of 0.1 g/mL egg-white lysozyme (Sigma-Aldrich) was added to the suspension, the resultant suspension was stirred and treated for 1 hour on ice. Furthermore, 5 μL, of 1 mol/L magnesium acetate containing 1 mol/L MgCl₂ was added to the suspension, which was then centrifuged at 6,500×g and 4° C. for 10 minutes to collect spheroplast. The collected spheroplast was suspended in 50 mL of a 20 mmol/L HEPES buffer solution (pH7.6) containing 0.1 mmol/L magnesium acetate and 0.9% NaCl, and the resultant suspension was allowed to stand still for 5 minutes on ice and thereafter centrifuged to wash. A bacteriolytic reagent was added to a precipitate of the spheroplast after the washing, and the resultant mixture was vigorously stirred at 4° C. Thus, bacteriolysis was performed. As the bacteriolytic reagent, 4 mL of a 20 mmol/L HEPES buffer solution (pH7.6) containing 100 μg/mL tRNA (derived from Saccharomyces cerevisiae, Sigma-Aldrich), 0.1% human serum albumin (Sigma-Aldrich), 50 units of RNase A inhibitor (TOYOBO CO., LTD.), 210 units of DNase (Invitrogen), 1/2 pieces of complete mini EDTA-free proteinase inhibitor cocktail tablets (Roche Ltd.), 0.5% TritonX-100, and 0.1 mmol/L magnesium acetate was used. The bacterial lysate was aspirated and discharged with a syringe provided with a 27-gauge injection needle, and a disruption of spheroplast was accelerated, and a genomic DNA was sheared. Thereafter, the resultant mixture was stood still for 5 minutes on ice and centrifuged at 17,000×g for 10 minutes to collect a supernatant. This supernatant was used as a bacterial lysate.

(2) Recovery of RNA from Complex

Selection beads were prepared as follows in advance. First, 20 μL of Polybead polystyrene 1.0-micron microsphere (Polysciences, Inc) was washed three times with 1 mL of a 0.1 mol/L borate buffer solution (pH8.5) and was suspended in 40 μL of a 0.1 mol/L borate buffer solution (pH8.5) containing 400 μg/mL human intelectin-1. This suspension thus obtained was incubated at room temperature for 18 hours while shaking and was thereafter centrifuged to collect the beads. The beads wore suspended in 100 μL of a 0.1 mol/L borate buffer solution (pH8.5) containing 10 mg/mL human serum albumin. The suspension thus obtained was then incubated at room temperature for 30 minutes while shaking and thereafter centrifuged to collect beads. This operation was repeated a total of three times. Then, the collected beads were suspended and stored in 40 μL of a 20 mmol/L HEPES buffer solution (pH7.6) containing 10 mg/mL human serum albumin and 0.9% NaCl. The beads were used as selection beads.

Subsequently, 1.5 to 4 mL of the bacterial lysate was added to 3 μL of the selection beads. The resultant mixture was then stirred at 4° C. for 30 minutes and was thereafter centrifuged at 17,000×g and 4° C. for 5 minutes to collect the beads. The collected beads were centrifuged and washed four times with a 20 mmol/L HEPES buffer solution (pH7.6) containing 0.5% TritonX-100 and 0.1 mmol/L magnesium acetate. 150 μL of a Trizol reagent (trade name, Invitrogen) was then added thereto, a resultant mixture was allowed to stand still at room temperature for 5 minutes. Thereafter, a soluble fraction including mRNA was collected using Ultrafree (0.22 μm, Millipore Corporation). The soluble fraction was subjected to chloroform extraction to collect an aqueous layer. 1 μL of Ethatinmate (trade name, NIPPON GENE CO., LTD.) was added to the aqueous layer, which was then subjected to isopropanol precipitation. A precipitate thus obtained was treated at 37° C. for 30 minutes using 5 units of DNase I (Promega KK.) and was then subjected to phenol-chloroform extraction and ethanol precipitation. Thus, purified RNA was obtained.

### (3) Construction of Novel Library

RT-PCR was performed by One step RT-PCR kit (trade name, QIAGEN) using a half amount of the purified RNA. The conditions include an annealing temperature of 57° C. and the number of cycles of 20. As primers, the following two kinds of primers were used in combination (SEQ ID NOs: 77 and 78).

Primer D1
(SEQ ID NO: 77)
CGGAAGACACGGCCGTTTATTACGC
Primer D2
(SEQ ID NO: 78)
TCTAGATTAGCGGCCGCTGGAAACG

After 20 cycles of the RT-PCR, 100 μmol/L primer D1 and 100 μmol/L probe D2 each with an amount which is 1/10 of the amount of the reaction solution were added to the reaction solution obtained after the RT-PCR. The reaction solution was heated at 95° C. for 30 seconds and at 94° C. for 3 minutes, and thereafter, a cycle of treatment at 57° C. for 1 minute and at 72° C. for 1 minute was repeated a total of five times. After the reaction, the reaction solution was subjected to ethanol precipitation to collect DNA. The DNA was treated at 37° C. for 3 hours using 25 units of exonuclease I (Takara Bio Inc.) to digest an excess amount of primers. The collected DNA was subjected to phenol-chloroform extraction and ethanol precipitation, and a double-stranded DNA thus obtained was dissolved in 10 μL of TE. 1 μL of this DNA solution was digested at 37° C. for 7 hours using 15 units of PstI and 15 units of NotI and thereafter subjected to phenol-chloroform extraction and ethanol precipitation to collect a DNA fragment. The DNA fragment thus obtained as it was or the DNA fragment thus obtained after being treated with alkaline phosphatase (calf intestine) (Takara. Bio Inc.) and being subjected to phenol-chloroform extraction and ethanol precipitation was used as a selected fragment.

The ⅓ amount of the selected fragment and 1 μg of the vector for library (HTX-VHH-shot/pColdv1) produced in the item “3, (3-2)” in Example 1 were mixed, the resultant mixture was subjected to a ligation reaction at 14° C. for 18 hours using 1750 units of T4 DNA ligase (Takara Bio Inc.). The reaction was performed in a 6 mmol/L, tris buffer solution (pH7.5) containing 0.1 mg/mL Bovine serum albumin, 7 mmol/L 2-mercaptoethanol, 0,1 mmol/L ATP, 2 mmol/L dithiothreitol, 1 mmol/L spermidine, 5 mmol/L NaCl, and 6 mmol/L MgCl₂. Thus, the selected fragment as the insert for library was inserted into the vector for library, and a library vector including a random region inserted thereinto was newly constructed,

10 μg of tRNA (derived from Saccharomyces cerevisiae, Sigma-Aldrich) was added to the reaction solution, and the resultant mixture was subjected to phenol-chloroform extraction and ethanol precipitation and was then dissolved in 5 μL of TE. The whole amount of the solution thus obtained was mixed in Escherichia coli to transform. The conditions of the transformation were the same as in Example 1. The transformed Escherichla coli was suspended in 3 mL of SOC, and the resultant suspension was then subjected to shaking culture at 37° C. for 1 hour, and thereafter, ampicillin was added thereto so as to have a final concentration of 100 μg/ml. Subsequently, the resultant suspension was subjected to shaking culture at 37° C. for 5 hours. 0.21 mL DMSO was added to this culture solution thus obtained, which was then mixed. The resultant culture solution was frozen in liquid nitrogen and stored at −80° C. immediately after the mixture.

The above-described steps of constructing each library vector, collecting each HTX-HVV protein, collecting each RNA binding to the protein, amplifying each DNA including a random region, fragmentating each DNA amplification product, and inserting each DNA fragment into a vector for library were repeated a total of two to three times. Then, a part of the transformed Escherichla coli was inoculated into a LB plate containing 50 μg/mL ampicillin to cause a colony to be formed. The colony thus obtained was inoculated in 0.5 mL of a LB medium containing 100 μg/mL ampicillin, which was then subjected to shaking culture at 37° C. for 8 hours. In the culture, a 96-well deep plate (Thermo Fisher Scientific K.K.) was used. The culture solution thus obtained was cooled at 10° C. for 30 minutes, and IPTG was then added thereto so as to have a final concentration of 0.5 mmol/L. The resultant mixture was subjected to shaking culture at 10° C. for 18 hours. This culture solution thus obtained was centrifuged at room temperature for 15 minutes to collect bacterial cells. Then, the bacterial cells were frozen in liquid nitrogen. The frozen bacterial cells were melted at room temperature, thereafter 200 μL of a 10 mmol/L HEPES buffer solution (pH7.6) containing 5,000 units/mL rLysozyme (Merck), 12.5 units/mL Benzonase (Merck), 0.5% TritonX (registered trademark)-100, and 1 mmol/L EDTA was added thereto, and the resultant mixture was then incubated at room temperature for 15 minutes while shaking. Thus, bacteriolysis was performed. This mixture thus obtained was frozen using liquid nitrogen and then re-melted. Thereafter, magnesium acetate with a final concentration of 2.5 mmol/L was added thereto, which was then stirred. Subsequently, the mixture thus obtained was allowed to stand still at room temperature for 10 minutes. Thus, bacterial lysate was obtained.

### (4) Screening

The bacterial lysate was diluted 2-hold with a 20 mmol/L tris buffer solution (pH7.6) containing 0.1% Tween-20, 0.2% Bovine serum albumin (Sigma-Aldrich), and 0.9% NaCl. For screening, this diluted bacterial lysate was used.

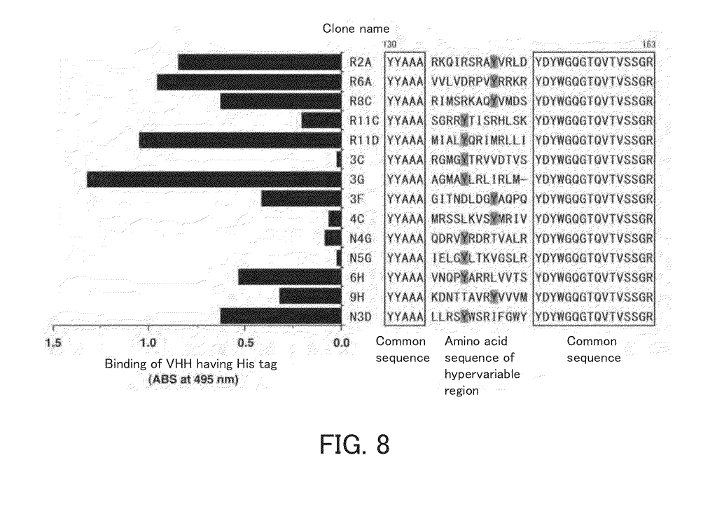

Binding clones were screened by ELISA shown below. First, 50 μL per a well of a 50 mmol/L carbonate buffer solution (pH9.0) containing 1 μg/mL human intelectin-1 as an antigen was added to a 96-well plate (ASAHI GLASS CO., LTD.), which was then allowed to stand still at room temperature for 3 hours to cause the antigen to be absorbed. Thereafter, each well was blocked with 200 μL of a 20 mmol/L tris buffer solution (pH7.6) containing 1% Bovine serum albumin (Sigma-Aldrich) and 0.9% NaCl, 50 μL of the diluted bacterial lysate was added to the well, which was then cultured at room temperature for 1.5 hours. The well was washed four times with a tris buffer solution (pH7.6) containing 0.1% Tween-20 and 0.9% NaCl. Thereafter, 50 μL, of a 20 mmol/L tris buffer solution (pH7.6) containing horseradish peroxidase-labeled anti-His tag antibody (QIAGEN) diluted 2000-hold, 0.2% Bovine serum albumin, and 0.9% NaCl was added to the well, which was then reacted at room temperature for 1 hour. The well was washed four times with a tris buffer solution (pH7.6) containing 0.1% Tween-20 and 0.9% NaCl, and then, Step Ultra TMB-ELISA (trade name, Thermo Fisher Scientific K.K.) was added to the well to cause a coloring reaction to be performed. The reaction was terminated with sulfuric acid, and thereafter, an absorbance at 450 nm was measured. Positive clones were sequenced, and amino acid sequences of the binding clones each exerting a binding property were determined. Amino acid sequences of variable regions of representative clones among the obtained positive clones and the results of the ELISA thereof are shown in FIG. 8.

In FIG. 8, a graph on the left side shows binding strengths to human intelectin-1 in HTX-HVV proteins expressed from the respective clones. Random regions and surrounding amino acid sequences thereof expressed from the respective clones are shown on the right side of FIG. 8. The amino acid sequences are represented by sequence numbers in order from the top. According to the screening method, peptide exerting a binding property to an antigen can be screened simply by repeating the above-mentioned steps. Thus, according to the present invention, for example, peptide including a variable region exerting a binding property to an antigen can be obtained without an immunizations into animals which is a conventional way, and based on the peptide, a human antibody and the like can be easily designed, for example.

### Example 3

Screening for a variable region binding to human TNF-α was performed using the HTX-VHH-shot/pColdv1 produced in Example 1.

Clones which specifically bind to human TNF-α were obtained by a treatment in the same manner as in Example 2 except that human TNF-α (Pepro Tech Inc.) was used as a substitute for human intelectin-1 in production of selection beads, and the following primer D3 was used as a substitute for the primer D2 as a primer for RT-PCR.

Primer D3
(SEQ ID NO: 79)
CTAGTAGCGGCCGCTTATCTACCGCTGGAAACGGTCACCTGGGT

The results of these are shown in Table 13 below. In Table 13, A to H vs 1 to 12 each show a well number in a plate, and a value in each cell show a measurement value Obtained by ELISA as in Example 2. As shown in Table 13, according to the present invention, a clone exerting a binding property to an antigen can be selected from the measurement values obtained by ELISA in the respective wells of the plate.

TABLE 13

| | 1 | 2 | 3 | 4 | 5 | 6 | 7 | 8 | 9 | 10 | 11 | 12 |
|---|---|---|---|---|---|---|---|---|---|---|---|---|
| A | 1.102 | 0.375 | 0.089 | 0.040 | 0.044 | 0.693 | 0.624 | 0.004 | 2.065 | 0.007 | 0.026 | 1.915 |
| B | 0.050 | 0.113 | 0.063 | 0.030 | 0.047 | 0.180 | 0.041 | 0.023 | 0.377 | 0.892 | 0.031 | 0.042 |
| C | 0.390 | 0.027 | 0.067 | 0.025 | 0.178 | 0.068 | 0.019 | 0.239 | 1.709 | 0.044 | 0.162 | 1.438 |
| D | 0.802 | 0.233 | 0.046 | 0.532 | 0.067 | 0.230 | 0.058 | 0.007 | 0.168 | 0.033 | 1.000 | 0.000 |
| E | 0.101 | 1.635 | 0.409 | 0.172 | 0.035 | 0.048 | 0.252 | 0.044 | 0.492 | 0.041 | 0.074 | 0.114 |
| F | 0.037 | 0.373 | 0.463 | 0.229 | 0.104 | 0.032 | 0.150 | 0.070 | 0.172 | 0.220 | 0.063 | 0.542 |
| G | 0.036 | 0.160 | 0.036 | 0.042 | 0.510 | 0.168 | 0.030 | 1.731 | 0.026 | 0.031 | 0.096 | 1.315 |
| H | 0.041 | 0.257 | 0.030 | 0.267 | 0.043 | 0.031 | 0.057 | 0.102 | 1.750 | 0.222 | 0.097 | 0.074 |

### Example 4

Screening for a variable region binding to human intelectin-1 was performed using the HTX-VHH-shot/pColdv1 produced in Example 1

Clones which specifically bind to human intelectin-1 were obtained by a treatment in the same manner as in Example 2 except that the complementary oligonucleotide 92 (SEQ ID NO: 74) was used as a substitute for the complementary oligonucleotide B1 (SEQ ID NO: 73) in preparation of an insertion for library, and the following primer D4 was used as a substitute for the primer D2 as a primer for RT-PCR.

Primer D4
(SEQ ID NO: 80)
CACTTAGCGGCCGCTCACGTAGGC

The results of these are shown in Table 14 below. In Table 14, A to H vs 1 to 12 each shows a well number, and a value in each cell shows a measurement value obtained by ELISA as in Example 2. As shown in Table 14, according to the present invention, a clone exerting a binding property to an antigen can be selected from the measurement values obtained by ELISA in the respective wells of the plate.

TABLE 14

| | 1 | 2 | 3 | 4 | 5 | 6 | 7 | 8 | 9 | 10 | 11 | 12 |
|---|---|---|---|---|---|---|---|---|---|---|---|---|
| A | 0.420 | 0.032 | 0.322 | 1.072 | 0.225 | 0.748 | 0.335 | 0.687 | 1.114 | 0.028 | 0.039 | 0.495 |
| B | 0.973 | 1.111 | 0.310 | 0.383 | 1.171 | 0.225 | 0.413 | 0.387 | 0.884 | 0.128 | 0.395 | 0.485 |
| C | 1.313 | 0.830 | 0.013 | 1.166 | 0.638 | 1.546 | 0.466 | 0.265 | 0.159 | 0.887 | 1.127 | 0.884 |
| D | 0.451 | 0.339 | 0.806 | 1.316 | 1.472 | 0.527 | 1.018 | 0.778 | 0.892 | 1.474 | 0.035 | 1.209 |
| E | 1.027 | 1.639 | 0.658 | 0.447 | 1.418 | 0.003 | 0.921 | 0.294 | 0.935 | 0.516 | 0.862 | 0.000 |
| F | 0.375 | 0.262 | 0.793 | 0.584 | 0.292 | 0.485 | 0.770 | 0.401 | 0.472 | 0.658 | 0.255 | 0.540 |
| G | 0.006 | 1.413 | 0.033 | 1.314 | 0.087 | 0.353 | 0.920 | 0.103 | 0.981 | 0.181 | 0.031 | 0.766 |
| H | 0.868 | 1.003 | 0.379 | 0.410 | 0.029 | 0.650 | 0.458 | 0.719 | 0.876 | 0.779 | 0.082 | 0.359 |

As described above, according to the present invention, by utilizing the binding between the peptide tag and the aptamer corresponding thereto, the information on the encoding nucleic acid of the antibody candidate that binds to the antigen can be easily analyzed, and the amino acid sequence of the antibody candidate can be determined based on the analysis result. Therefore, according to the present invention, for example, the selection of the antibody candidate can be performed without causing enormous time and efforts unlike the obtainment of the antibody by immunization or the like.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

This application is based upon and claims the benefit of priority from Japanese patent application No. 2011-215049, filed on Sep. 29, 2011, the disclosure of which is incorporated herein its entirety by reference.

### INDUSTRIAL APPLICABILITY

According to the nucleic acid construct of the present invention, a complex of the fusion transcript and the fusion translation product can be formed by utilizing the binding between the transcribed aptamer and the translated peptide tag. In the complex, the fusion transcript includes the transcript of the any peptide-encoding sequence, and the fusion translation product includes the any peptide. Therefore, in the case where the complex binds to an antigen, for example, by the identification of the transcript in the complex, the antibody candidate that is bindable to the target and the any peptide can be identified. In this manner, according to the present invention, simply by inserting the any peptide-encoding nucleic acid into the first nucleic acid construct according to the present invention to form the complex and recovering the complex that is bound to the antigen, the antibody candidate that is bindable to the antigen and an encoding nucleic acid of the antibody candidate can be identified easily. Accordingly, the present invention provides a very useful tool and method for screening for a novel antibody to an antigen, for example, in medical fields.

1. A nucleic acid construct for expressing an antibody candidate to an antigen, the nucleic acid construct comprising the following encoding nucleic acids (x) to (z):
(x) an encoding nucleic acid of an antibody candidate, obtained by inserting an encoding nucleic acid of any peptide into an encoding nucleic acid of a variable region of an antibody;
(y) an encoding nucleic acid of a peptide tag; and
(z) an encoding nucleic acid of a nucleic acid molecule that binds to the peptide tag, wherein
the encoding nucleic acids (x), (y), and (z) are bound with one another so that the encoding nucleic acids (x), (y), and (z) are transcribed as a fusion transcript, and the encoding nucleic acids (x) and (y) are translated as a fusion translation product.

2. The nucleic acid construct according to claim 1, wherein
the encoding nucleic acid of any peptide is inserted into an encoding nucleic acid of a hypervariable region in the variable region.

3. The nucleic acid construct according to claim 1 or 2, wherein
the variable region is a variable region VHH derived from Camelidae

4. The nucleic acid construct according to claim 3, wherein
the encoding nucleic acid of any peptide is inserted into an encoding nucleic acid of a CDR3 region in the variable region VHH.

5. The nucleic acid construct according to any one of claims 1 to 4, wherein the nucleic acid construct is a vector.

6. The nucleic acid construct according to claim 5, wherein
the vector is a cold-shock expression vector.

7. The nucleic acid construct according to any one of claims 1 to 6, wherein
the peptide tag is a histidine tag.

8. The nucleic acid construct according to claim 7, wherein
the nucleic acid molecule that binds to the peptide tag includes any of the following polynucleotides (a) to (d):
(a) polynucleotide that includes a base sequence represented by SEQ ID NO: 17:
(SEQ ID NO: 17)
GGUNₙAYUₘGGH,
where, N represents A, C, U. T, n of N, represents the number of Ns, which is an integer of 1 to 3, Y represents U, T, or C, m of Uₘ represents the number of Us, which is an integer of 1 to 3, and H represents U, T, C, or A;
(b) polynucleotide that includes a base sequence obtained by substitution, deletion, addition, and/or insertion of one or more bases in the base sequence in the polynucleotide (a) and binds to the histidine tag;
(c) polynucleotide that includes a base sequence represented by SEQ ID NO: 18:
(SEQ ID NO: 18)
GGCGCCUUCGUGGAAUGUC;
and
(d) polynucleotide that includes a base sequence Obtained by substitution, deletion, addition, and/or insertion of one or more bases in the base sequence in the polynucleotide (c) and binds to the histidine tag.

9. The nucleic acid construct according to any one of claims 1 to 8, wherein
the encoding nucleic acid of a peptide tag (y), the encoding nucleic acid of an antibody candidate (x), and the encoding nucleic acid of a nucleic acid molecule (z) are arranged in this order.

10. The nucleic acid construct according to any one of claims 1 to 9, wherein
the encoding nucleic acid of a peptide tag (y), the encoding nucleic acid of an antibody candidate (x), and the encoding nucleic acid of a nucleic acid molecule (z) are arranged from 5′ to 3′ in this order.

11. A method for screening for an antibody or an encoding nucleic acid of the antibody using the nucleic acid construct according to any one of claims 1 to 10, the method comprising the following steps (A) to (C):
(A) a step of expressing the nucleic acid construct to form a complex of a fusion transcript obtained by transcribing the encoding nucleic acid of an antibody candidate (x), the encoding nucleic acid of a peptide tag (y), and the encoding nucleic acid of a nucleic acid molecule (z) and a fusion translation product obtained by translating the encoding nucleic acid of an antibody candidate (x) and the encoding nucleic acid of a peptide tag (y);
(B) a step of brining the complex and an antigen into contact with each other; and
(C) a step of recovering the complex binding to the antigen.

12. The method according to claim 11, wherein in the step (B),
the antigen is an antigen immobilized on a solid phase,

13. The method according to claim 11 or 12, further comprising the step (D):
(D) a step of synthesizing an encoding nucleic acid of any peptide in the antibody candidate, using the fusion transcript in the complex as a template.

14. The method according to claim 13, wherein
a nucleic acid construct according to any one of claims 1 to 10 including the encoding nucleic acid of the any peptide inserted thereinto is newly prepared using the encoding nucleic acid of the any peptide obtained in the step (D), and the steps (A), (B), and (C) are again performed.

15. The method according to claim 13 or 14, wherein
the steps (A) to (D) are performed repeatedly.

16. The method according to any one of claims 13 to 15, wherein
a base sequence of the encoding nucleic acid of the any peptide obtained in the step (D) is determined.

17. The method according to claim 16, wherein
an amino acid sequence of the any peptide is determined from the base sequence of the encoding nucleic acid of the any peptide.

18. The method according to any one of claims 11 to 17, wherein
the nucleic acid construct is expressed in vitro.

19. . The method according to claim 17, wherein
the nucleic acid construct is expressed in Escherichia coli.
